(19) United States
(12) Patent Application Publication
(10) Pub. No.: US 2008/0299104 A1
(43) Pub. Date: Dec. 4, 2008

(54) METHODS FOR DETECTING AGENTS INVOLVED IN NEURONAL APOPTOSIS AND COMPOSITIONS THEREOF

(75) Inventors: Mary B. Kennedy, Pasadena, CA (US); Irene Knuesel, Zurich (CH); Jeong Oh, Thousand Oaks, CA (US)

Correspondence Address: Lisa A. Haile, J.D., Ph.D.;DLA PIPER US LLP, Suite 1100, 4365 Executive Drive, San Diego, CA, 92121-2133, US

(73) Assignee: CALIFORNIA INSTITUTE OF TECHNOLOGY

(21) Appl. No.: 12/126,691

(22) Filed: May 23, 2008

Related U.S. Application Data
(62) Division of application No. 11/174,201, filed on Jul. 1, 2005.
(60) Provisional application No. 60/585,477, filed on Jul. 1, 2004.

(51) Int. Cl. A61K 38/45; A61K 38/00; A61K 31/7088; A61P 25/00

(52) U.S. Cl. 424/94.5; 514/2; 514/44

(57) ABSTRACT

The invention provides methods of detecting and identifying agents which moduclate the expression or activity of synGAP, a brain-specific Ras/Rap GTPase activating protein. In vivo studies using knock-out and conditional knock-out transgenic animals show that the level of apoptosis in neurons correlates inversely with the level of synGAP protein, indicating that neuronal apoptosis is enhanced by reduction of synGAP. The invention also describes that synGAP is capable of modulating signal transduction pathways associated with the NMDA receptor and triggering apoptosis, including activation of Ras-GAP. Phosphorylation of synGAP by CaMKII increases its Ras GTPase-activating activity by about 70-95%. Moreover, when these and other phosphorylation sites in the synGAP carboxyl tail are mutated, stimulation of GAP activity after phosphorylation is reduced to about 21±5% as compared to about 70-95% for the wild type protein. Also, phosphosite-specific antibodies used to determine levels of synGAP phosphorylation are described herein.

### CROSS REFERENCES TO RELATED APPLICATIONS

This application is a divisional application of U.S. application Ser. No. 11/174,201 filed Jul. 1, 2005, currently pending, which claims the benefit of priority under 35 U.S.C. § 119(e) of U.S. Application Ser. No. 60/585,477, filed Jul. 1, 2004. The disclosure of each of the prior applications is considered part of and is incorporated by reference in the disclosure of this application.

### GOVERNMENT SUPPORT

This invention was made in part with government support under NIH grants NS17660 and NS28710 and Swiss National Foundation fellowship 823A-064694.

### BACKGROUND INFORMATION

1. Field of the Invention

The invention relates generally to neuronal cell death or apoptosis, and more specifically to, modulation of the expression or activity of synGAP, including activation of Ras-GTPase.

2. Background of the Invention

Cell death can be categorized morphologically as necrosis or apoptosis. Necrosis, is characterized by cellular swelling, rupture of plasma and internal membranes, and eventual leakage of cellular contents into the extracellular space. In contrast, apoptosis, involves progressive condensation of cytoplasm and nuclear chromatin and eventual fragmentation or blebbing of cellular membranes into apoptotic bodies, which are then digested by macrophages or adjacent epithelial cells. Necrosis typically occurs by certain pathological conditions, e.g., injury by complement, hypoxia, or exposure to a variety of toxins. Apoptosis is programmed cell death as signaled by the nuclei in normally functioning human and animal cells (e.g., age, state of cell health and like conditions). Programmed cell death is a concept that certain cells are determined to die at specific stages and specific sites during development, for example, cells in the spaces between the developing digits of vertebrates, thus dividing them.

Apoptosis is a fundamental aspect of normal development in invertebrates and vertebrates, vertebrate tissue homeostasis, and plays a role in the underlying pathological mechanism in disorders which involve cell death, including degenerative disorders, e.g., stroke, traumatic brain injury, and myocardial infarction, conditions associated with aging (e.g., Alzheimer's disease, Parkinson's disease and the like), viral and other types of infection, and even some cancers. Apoptosis appears to be genetically regulated, however, apoptosis can also be induced by exposing cells to radiation, heat, cytotoxic agents, and abnormal changes in cellular biology. The mitochondria may also be involved in apoptosis. Excessive cell death may result in crippling degenerative disorders, for example, the elimination of neurons, and other cell types, following ischemia and reperfusion; the destruction of cells after exposure to ionizing or ultraviolet radiation in the treatment of neoplastic disorders; and the annihilation of vital CD4+ T-lymphocytes in HIV (human immunodeficiency virus) infected patients. These disorders are thought to stem from ectopically programmed cell death, e.g., metabolic or infective factors that induce the apoptosis. Also, too little cell death can result in proliferative disorders, such as neoplastic disorders or autoimmune disease when a particular immune cell lives beyond its appropriate life span. Thus, it is uncertain whether the morphological classification of cell death reflects real differences in the underlying physiological mechanisms of cell death.

Although the initiation and morphology of cell death vary, there is evidence which suggests that most physiological and some pathological cell deaths may share a common feature involving the activation or inactivation of cell death genes. Thus, a better understanding of the mechanisms of cell death would have wide biological application and provide a basis for altering or controlling the process.

### SUMMARY OF THE INVENTION

The present invention relates to methods modulating the expression or activity of synGAP in a cell.

In one embodiment of the invention, there is provided a method for modulating the expression or activity of synGAP in a cell by contacting the cell with a test agent and detecting a change in expression or activity of synGAP in the presence of the test agent as compared to in the absence of the test agent, wherein a difference identifies a test agent as an agent that modulates the expression or activity of synGAP in the cell.

In one embodiment of the invention, there is provided a method of treating a subject having apoptotic cell death disorder by administering to a subject in need thereof, or to cells of a subject, a therapeutically effective amount of an agent that increases expression of synGAP or increases synGAP activity. In one aspect, the agent increases RasGTPAse activity of synGAP of a subject having a disorder selected from Alzheimer's disease, Parkinson's disease, Huntington's disease or amyotropic lateral sclerosis (ALS).

In another embodiment of the invention, there is provided a method of ameliorating a pathologic disorder in a subject including administering an agent to the subject, wherein the agent increases the expression of synGAP in a cell thereby ameliorating the disorder. In one aspect, the disorder is a degenerative disorder, or a neurodegenerative disorder including Alzheimer's disease, Parkinson's disease, Huntington's disease or amylotropic lateral sclerosis.

Yet, in another embodiment of the invention, there is provided methods to produce an antibody that binds synGAP polypeptides or functional fragments of the polypeptide thereof. For example, in one embodiment of the invention, there is provided methods to produce phospho site-specific antibodies which bind to phosphorylated synGAP peptides, in particular, phosphorylated synGAP peptides at specific serine amino acid residues (e.g., 750, 751, 765, 764, 765, 1058, 1123 and any combinations thereof) of synGAP. In another embodiment of the invention, there is provided non-phosphorylated site-specific antibodies, in particular, synGAP peptides at specific serine amino acid residues (e.g., 750, 751, 765, 764, 765, 1058, 1123 and any combinations thereof) of synGAP.

In another embodiment of the invention, there is provided a method of conferring neuroprotection on a population of cells by providing an agent which increases synGAP expression or activity and administering a therapeutically effective amount of the agent to the population of cells so as to confer neuroprotection. In one aspect, the agent, for example, CaMKII, increases synGAP activity thereby altering RasGTPase activity.

Still, in another embodiment of the invention, there is provided a transgenic non-human animal having a transgene disrupting expression of synGAP, chromosomally integrated into the germ cells of the animal, whereby the animal has a phenotype of reduced synGAP protein as compared with a wild-type animal.

Yet, in another embodiment of the invention, there is a provided a method for producing a transgenic non-human animal having a phenotype characterized by a conditional or reduced expression of synGAP polypeptide, the method by introducing at least one transgene into a zygote of an animal, the transgene(s) including a DNA construct encoding a selectable marker; transplanting the zygote into a pseudopregnant animal; allowing the zygote to develop to term; and identifying at least one transgenic offspring whose genome comprises a disruption of the endogenous synGAP polynucleotide sequence by the transgene.

### BRIEF DESCRIPTION OF THE DRAWINGS

FIG. 1 is a graph showing the stoichiometry of phosphorylation of synGAP by CaMKII. Recombinant synGAP (2 μg) in a Hi-5 membrane fraction (solid line) was phosphorylated in the presence of exogenous purified CaMKII (3 μg). Native synGAP (2.6 μg) in the postsynaptic density (PSD) fraction (dashed line) was phosphorylated with the endogenous CaMKII present in the PSD fraction (total PSD protein 50 μg).

FIG. 2 is a graph showing the effect of phosphorylation of synGAP by CaMKII on Ras GAP activity. RasGAP activity was assayed before and after phosphorylation of synGAP by CaMKII as described herein (see Example 1).

FIG. 3 is a graph showing that the increase in synGAP's GAP activity depends on CaMKII: (A) Dependence on amount of CaMKII in the phosphorylation reaction; (B) Effect of antibodies that inhibit CaMKII on the increase in GAP activity; (▪), phosphorylation in the absence of CaCl2 and calmodulin; (▴), phosphorylation in the presence of CaCl2 and calmodulin.

FIG. 4 is a graph showing the tryptic phosphopeptides generated from the GST-ctSynGAP fusion protein after phosphorylation by CaMKII; ( [symbol] ), acetonitrile concentration.

FIG. 5 shows mass spectrographs of phosphopeptides from the GST-ctSynGAP fusion protein.

FIG. 6 is a graph showing tryptic phosphopeptides generated from recombinant synGAP missing sites S1058 and S1123 after phosphorylation by CaMKII; [symbol] acetonitrile concentration.

FIG. 7 shows mass spectrographs of phosphopeptides from synGAP mutant missing sites 1058 and 1123.

FIG. 8 is a graph showing the loss of regulation of GAP activity in phosphosite mutants of synGAP.

FIG. 9 is a graph showing that phosphorylation at amino acid positions 765 and 1123 is regulated in vivo by activation of NMDA-type glutamate receptors: (A) Antisera against synthetic phosphopeptides with the sequence surrounding serine 765 or serine 1123 in synGAP on immunoblots of nonphosphorylated (−) and phosphorylated (+) postsynaptic density proteins. Arrows indicate the top of the gel and the position of synGAP; and (B) Cultures of cortical neurons prepared as described herein, exposed to 25 μM N-methyl-D-aspartate or to vehicle for 15 seconds, as described herein. The area shown in FIG. 9A includes proteins at the top of the gel down to MW about 96 kDa. Arrows indicate the top of the gel and the position of synGAP.

FIG. 10 is a graph showing the location of phosphorylation sites in synGAP. The right bracket indicates the boundaries of the disordered region predicted by the PONDRs software. The left bracket indicates the boundaries of the tandem repeats containing the phosphorylation sites between residues 750 and 765.

FIG. 11 is an illustration showing the construction of a synGAP knockout mouse: (A) The targeting construct included a Neo cassette flanked by Lox P sites inserted into intron 3 of the synGAP gene, and an additional downstream Lox P site within intron 9; (B) Immunoblot comparing synGAP expression levels during hippocampal and cortical development in wt; (C) Immunoblot confirming that all four isoforms of SynGAP protein are absent in brain extracts of ko mice (top) and are reduced in heterozygote (het) mice at P1 compared to wt.

FIG. 12 are graphs showing apoptotic cells/section (mean±SEM), visualized by staining with anti-activated Caspase-3 discussed herein, in sections of hippocampus, cerebellum, and cortex from wt and synGAP ko mice at E18, P0, and P1; *P<0.05, ***P<0.001 (unpaired Student's t-test).

FIG. 13 is an immunoblot of hippocampal extracts of wt, het, and ko litter mates on P1 with antibody against activated caspase-3 and GFAP; Scale bar: 20 μm.

FIG. 14 is a graph showing two distinct phenotypes of synGAP conditional mutant mice; cond-ko's, 4 (▪), normal cond-ko's (▴), controls ( [symbol] ).

FIG. 15 shows levels of synGAP protein in brains of synGAP cond-ko's and heterozygous controls at two weeks after birth. (A) Representative immunoblots with anti-synGAP antibody of hippocampal and cortical extracts of mini's, normal cond-ko, and control mice. Protein samples were run as duplicates. (B) Quantitative analysis of immunoblots was carried out as described under Materials and Methods. Densities of immunoreactive bands from cond-ko's (n=6) and mini's (n=3) were normalized to values for heterozygous controls (n=12; *p<0.05, **p<0.01, ***p<0.001).

FIG. 16 is an immunoblot and graph showing reduced levels of synGAP protein in 8 week old synGAP cond-ko mice. (A) Representative immunoblot of hippocampal and cortical extracts from heterozygous controls and cond-ko's with anti-synGAP antibody. Protein samples were run as duplicates. (B) Quantitative analysis of immunoblots reveals a statistically significant difference in levels of synGAP protein in hippocampus but not in cortex of 8 w old normal cond-ko mice compared to heterozygous control litter mates. Densities of bands in normal cond-ko samples (n=4) were normalized to those of heterozygous controls (n=4), **p<0.01.

### DETAILED DESCRIPTION

The present invention relates to methods of modulating the expression or activity of neuron-specific Ras GTPase-activating protein (synGAP) polypeptide protecting cells against apoptosis preventing cell death.

Some abbreviations used though-out the invention include but are not limited to: NMDA, N-methyl-D-aspartate; CaMKII, Ca²⁺/calmodulin-dependent kinase II; GAP, GTPase activating protein; wt, wild type; ko, synGAP knockout; flox, flanked by loxP sites; cond-ko, conditional synGAP knockout αCaMKII:crecre recombinase transgene controlled by the alpha CaMKII promoter.

SynGAP is about a 140 kDa protein in the postsynaptic density (PSD) fraction (Chen et al., 1998; Kim et al., 1998), and interacts with PSD-95 in a yeast two-hybrid screen (Kim et al., 1998). SynGAP RNA is detected only in the brain (Chen et al., 1998; Kim et al., 1998), and specifically only in neurons, including most excitatory neurons and a subset of inhibitory neurons (Zhang et al., 1997). Proteins in the PSD, including synGAP, are part of a signaling complex attached to the cytoplasmic tail of the N-methyl-D-aspartate-type glutamate (NMDA) receptor. SynGAP is a prominent substrate for calcium-calmodulin kinase III (CaMKII) in the PSD fraction, and phosphorylation by CaMKII produces greater than about 20%, in particular, about 75% increase in synGAP's Ras GAP activity. Hence, synGAP plays a crucial role in early development of the brain and in control of synaptic plasticity in the adult brain, as suggested by the phenotypes of mouse synGAP mutants, e.g., newborn mice with a deletion of synGAP die a few days after birth; whereas adult mice heterozygous for the deletion have altered synaptic plasticity (Komiyama et al., 2002; Kim et al., 2003).

Prior to the present invention, the role of synGAP in apoptosis was not well understood in cells. The invention provides for the first time an understanding of the role of synGAP and methods of treating a subject having apoptotic cell death disorder by administering an effective amount of an agent which modulates the expression or activity of synGAP. As used herein, the term “agent” describes any agonist, antagonist, peptide, peptidomimetic, antibody, or chemical, or pharmaceutical, including but not limited to, a nucleic acid (e.g., an aptamer, antisense RNA, or gene silencing RNA, e.g., a dsRNA, siRNA, stRNA, or RNA silencing hairpin), small molecules, mineral, protein, peptide, hormone, lipid, carbohydrate, vitamin, or co-enzyme, with the capability of altering or mimicking the physiological function or expression of a synGAP polypeptide.

When a disorder is associated with abnormal expression of synGAP (e.g., over expression, or expression of a mutated form of the protein, or inhibition as compared with a normal cell) or as a result of expression of a substrate for the synGAP polypeptide, a therapeutic approach which directly interferes with the translation of a synGAP polypeptide is possible. Alternatively, similar methodologies may be used to study gene activity. Accordingly, in one aspect, an agent which modulates the expression or activity of synGAP is an antisense nucleic acid molecule. An agent that is an antisense molecule may be directed to the structural gene region or to the promoter region of a synGAP gene. The agent may be an agonist, or antagonist, e.g., peptide, peptidomimetic, antibody, chemical, or molecule.

For example, antisense nucleic acid, double-stranded interfering RNA or ribozymes may be used to bind to a synGAP polypeptide mRNA sequence or to cleave it. Antisense RNA or DNA molecules bind specifically with a targeted gene's RNA message, interrupting the expression of that gene's protein product. The antisense binds to the messenger RNA forming a double stranded molecule which cannot be translated by the cell. Antisense oligonucleotides of about 15-25 nucleotides are easily synthesized and have an inhibitory effect just like antisense RNA molecules. In addition, chemically reactive groups, such as iron-linked ethylenediaminetetraacetic acid (EDTA-Fe) can be attached to an antisense oligonucleotide, causing cleavage of the RNA at the site of hybridization. Antisense nucleic acids are DNA or RNA molecules that are complementary to at least a portion of a specific mRNA molecule (Weintraub, Scientific American, 262:40, 1990). In the cell, the antisense nucleic acids hybridize to the corresponding mRNA, forming a double-stranded molecule. The antisense nucleic acids interfere with the translation of the mRNA, since the cell will not translate an mRNA that is double-stranded. Antisense oligomers of about 15 nucleotides are easily synthesized and are less likely to cause problems than larger molecules when introduced into the target synGAP polypeptide producing cell. The use of antisense methods to inhibit the in vitro translation of genes is well known in the art (Marcus-Sakura, Anal. Biochem., 172:289, 1988).

Therapeutic or prophylactic use of nucleic acids comprising at least six nucleotides that are antisense to the genes or cDNAs encoding an agent (e.g., a substrate) which modulates expression or activity of synGAP or portions thereof are encompassed by the invention herein. The antisense nucleic acids of the invention are double-stranded or single-stranded oligonucleotides, RNA, or DNA, or a modification or derivative thereof, and can be directly administered to a cell or produced intracellularly by transcription of exogenous, introduced sequences.

The antisense nucleic acids of the invention are of at least six nucleotides and are oligonucleotides ranging from 6 to about 50 oligonucleotides, in particular, at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. In addition, the antisense molecules may be polymers that are nucleic acid mimics, such as PNA, morpholino oligos, and LNA. Other types of antisense molecules include short double-stranded RNAs, short interfering RNAs (siRNAs), and short hairpin RNAs, and long dsRNA (>50 bp but usually <500 bp).

In one embodiment, the antisense molecule is an siRNA molecule. Hence, in one embodiment, synGAP expression or activity is inhibited by a short interfering RNA (siRNA) through RNA interference (RNAi) or post-transcriptional gene silencing (PTGS) (see, for example, Ketting et al., Genes Develop (2001) 15:2654-2659). siRNA molecules can target homologous mRNA molecules for destruction by cleaving the mRNA molecule within the region spanned by the siRNA molecule. Accordingly, siRNAs capable of targeting and cleaving homologous synGAP mRNA are useful for treating, for example, neurodegenerative diseases.

In another embodiment, agents which modulate the expression or activity of synGAP can include ribozymes. Ribozymes are RNA molecules possessing the ability to specifically cleave other single-stranded RNA in a manner analogous to DNA restriction endonucleases. There are two basic types of ribozymes namely, tetrahymena-type (Hasselhoff, Nature, 334:585, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are used over tetrahymena-type ribozymes for inactivating a specific mRNA species and 18-base recognition sequences are used as opposed to shorter recognition sequences.

The mechanism of ribozyme action involves sequence-specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage (for this sequence, see, e.g., U.S. Pat. No. 5,093,246). Ribozymes that cleave mRNA at site-specific recognition sequences can be used to destroy mRNA encoding synGAP. In a related embodiment, hammerhead ribozymes can be used. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art. The ribozymes of the present invention also include RNA endoribonucleases (hereinafter “Cech-type ribozymes”) such as the one that occurs naturally in Tetrahymena thermophila (known as the IVS, or L-19 IVS RNA). The Cech-type ribozymes have an eight base pair active site that hybridizes to a target RNA sequence where after cleavage of the target RNA takes place.

Through the modification of nucleotide sequences which encode these RNAs, it is possible to engineer molecules that recognize specific nucleotide sequences in an RNA molecule and cleave it (Cech, J. Amer. Med. Assn., 260:3030, 1988). A major advantage of this approach is that, because they are sequence-specific, only mRNAs with particular sequences are inactivated.

These and other uses of antisense and ribozymes methods to inhibit the in vivo translation of genes are known in the art (e.g., De Mesmaeker, et al., Curr. Opin. Struct. Biol., 5:343, 1995; Gewirtz, A. M., et al., Proc. Natl. Acad. Sci. U.S.A., 93:3161, 1996b; Stein, C. A., Chem. and Biol. 3:319, 1996).

Moreover, wherein any of the above agents is a nucleic acid molecule (e.g., antisense, aptamer, double-stranded RNA, siRNA and the like), the nucleic acid molecule may also include or encode a localization sequence to direct the agent to a particular cellular site by fusion to appropriate organellar targeting signals or localized host proteins. For example, a polynucleotide encoding a localization sequence, or signal sequence, can be used as a repressor and thus can be ligated or fused at the 5′ terminus of a polynucleotide encoding a polypeptide of the invention such that the localization or signal peptide is located at the amino terminal end of a resulting polynucleotide/polypeptide. The construction of expression vectors and the expression of genes in transfected cells involves the use of molecular cloning techniques also well known in the art. (See, for example, Sambrook et al., Molecular Cloning—A Laboratory Manual, 3rd Ed., Cold Spring Harbor Laboratory, Cold Spring Harbor, N.Y., 2001, and Current Protocols in Molecular Biology, M. Ausubel et al., eds., (Current Protocols, a joint venture between Greene Publishing Associates, Inc. and John Wiley & Sons, Inc., most recent Supplement)). These methods include in vitro recombinant DNA techniques, synthetic techniques and in vivo recombination/genetic recombination. (See also, Maniatis, et al., Molecular Cloning A Laboratory Manual, Cold Spring Harbor Laboratory, N.Y., 1989).

In another embodiment of the invention, the agent is an antibody. Production of antibodies and antibody fragments may be raised in various host animals immunized by injection with the agent which modulates the expression or activity of synGAP, or synGAP, or peptides thereof, including, truncated polypeptides, functional equivalents of the agent or mutated variant of the agent. Such host animals may include but are not limited to pigs, rabbits, mice, goats, and rats, to name but a few. Various adjuvants may be used to increase the immunological response, depending on the host species, including but not limited to Freund's adjuvant (complete and incomplete), mineral salts such as aluminum hydroxide or aluminum phosphate, chitosan, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and or coupling with molecules such as keyhole limpet hemocyanin, tetanus toxoid, diphtheria toxoid, ovalbumin, cholera toxin or fragments thereof. Polyclonal antibodies are heterogeneous populations of antibody molecules derived from the sera of the immunized animals.

A polyclonal or monoclonal antibody of the invention that binds to synGAP polypeptide is useful for the in vivo and in vitro detection of antigen. The invention herein describes phosphorylation site-specific antibodies and equivalent antibodies whereby the protein or peptide is not phoshphorylated, and their use in detecting the levels of phosphorylated and unphosphorylated protein in a sample. However, other uses of the antibodies are within the scope of the invention, for example, as a monoclonal antibody for treatment, to inhibit or activate synGAP expression or activity, or functional fragments thereof, or synGAP associated proteins or substrates. The concentration of a detectably labeled monoclonal antibody administered to a subject should be sufficient such that the binding to those cells, body fluid, or tissue having a synGAP polypeptide that is detectable compared to the background.

A monoclonal antibody of the invention can be detectably labeled using a radioisotope or other label. For in vivo diagnostic imaging, the type of detection instrument available is a major factor in selecting a given radioisotope. The radioisotope chosen must have a type of decay, which is detectable for a given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the host is minimized. Ideally, a radioisotope used for in vivo imaging will lack a particle emission, but produce a large number of photons in the 140-250 key range, which may be readily detected by conventional gamma cameras.

For in vivo diagnosis, radioisotopes may be bound to an immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the monoclonal antibodies of the invention are ¹¹¹In, ⁹⁷Ru, ⁶⁷Ga, ⁶⁸Ga, ⁷²As, ⁸⁹Zr, and ²⁰¹Tl.

The monoclonal antibodies of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements, which are particularly useful in such techniques, include ¹⁵⁷Gd, ⁵⁵Mn, ¹⁶²Dy, ⁵²Cr, and ⁵⁶Fe.

In another embodiment of the invention, an agent of the invention capable of modulating the expression or activity of synGAP, can be a polypeptide or protein. As used herein, a “polypeptide” or “protein” or equivalents thereof refers to a polymer in which the monomers are amino acid residues which are joined together through amide bonds. When the amino acids are alpha-amino acids, either the L-optical isomer or the D-optical isomer can be used, the L-isomers being typical. Examples of polypeptides useful in the methods and compositions of the invention include those polypeptides which modulate the expression or activity of synGAP, RasGTPase, or their functional fragments. Accordingly, the polypeptides of the invention are intended to cover naturally occurring proteins, as well as those which are recombinantly or synthetically synthesized. Hence, polypeptide or protein fragments are also encompassed by the invention. Fragments can have the same or substantially the same amino acid sequence as the naturally occurring protein. A polypeptide or peptide having substantially the same sequence means that an amino acid sequence is largely, but not entirely, the same, but retains a functional activity of the sequence to which it is related. In general polypeptides of the invention include peptides, or full length protein, that contains substitutions, deletions, or insertions into the protein backbone, that would still have an approximately 70%-90% homology to the original protein over the corresponding portion. A yet greater degree of departure from homology is allowed if like-amino acids, i.e. conservative amino acid substitutions, do not count as a change in the sequence

Agents of the invention may include polypeptides which are substantially related but for conservative variations, such polypeptides are encompassed by the invention. A conservative variation denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine. As used herein, the term “conservative variation” also includes the use of a substituted amino acid in place of an unsubstituted parent amino acid provided that antibodies raised to the substituted polypeptide also immunoreact with the unsubstituted polypeptide.

However, contemplated agents of the invention may have modifications and substitutions which are not limited to replacement of amino acids. For a variety of purposes, such as increased stability, solubility, or configuration concerns, one skilled in the art will recognize the need to introduce, (by deletion, replacement, or addition) other modifications. Examples of such other modifications include incorporation of rare amino acids, dextra-amino acids, glycosylation sites, cytosine for specific disulfide bridge formation. The modified peptides can be chemically synthesized, or the isolated gene can be site-directed mutagenized, or a synthetic gene can be synthesized and expressed in bacteria, yeast, baculovirus, and tissue culture and so on.

Once a polypeptide or peptide agent has been identified and tested, peptide analogs are commonly made. Peptide analogs are commonly used in the pharmaceutical industry as non-peptide drugs with properties analogous to those of the template peptide. These types of non-peptide compound are termed “peptide mimetics” or “peptidomimetics”. See Fauchere, 1986, Adv. Drug Res. 15:29; Veber & Freidinger, 1985, TINS p. 392; and Evans et al., 1987, J. Med. Chem. 30:1229, which are incorporated herein by reference for any purpose. Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH2-NH—, —CH2-S—, —CH2-CH2-, —CH═CH-(cis and trans), —COCH2-, —CH(OH)CH2-, and —CH2SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, constrained peptides comprising a consensus sequence or a substantially identical consensus sequence variation may be generated by methods known in the art (Rizo & Gierasch, 1992, Ann. Rev. Biochem. 61:387, incorporated herein by reference for any purpose); for example, by adding internal cysteine residues capable of forming intramolecular disulfide bridges which cyclize the peptide.

In one embodiment, small molecule entities or test compounds which may be useful in the present invention include compounds which may specifically interact with synGAP or agents which directly and indirectly modulate the expression or activity of synGAP. Examples of such molecules include, but are not limited to, drugs or therapeutic compounds, toxins, cytokines, and bioactive peptides.

Once an agent has been identified, the nucleotide sequence encoding the agent (e.g., polypeptide, peptide, antibody, and functional fragments thereof), may be inserted into a recombinant expression vector. A recombinant expression vector generally refers to a plasmid, virus or other vehicle known in the art that has been manipulated by insertion or incorporation of a nucleic acid sequences. For example, a recombinant expression vector of the invention includes a polynucleotide sequence encoding a synGAP polypeptide or having synGAP activity or a fragment thereof or encoding an APP fusion product or fragment thereof. The expression vector typically contains an origin of replication, a promoter, as well as specific genes which allow phenotypic selection of the transformed cells. Vectors suitable for use in the invention include, but are not limited to the T7-based expression vector for expression in bacteria (Rosenberg, et al., Gene 56:125, 1987), the pMSXND expression vector for expression in mammalian cells (Lee and Nathans, J. Biol. Chem. 263:3521, 1988), baculovirus-derived vectors for expression in insect cells, cauliflower mosaic virus, CaMV; tobacco mosaic virus, TMV.

In the present invention, nucleotide sequences encoding synGAP and synGAP functional fragments thereof, are inserted or incorporated into E. coli vectors. However, other vectors from other systems can also be used. For example, in yeast, a number of vectors containing constitutive or inducible promoters may be used. For a review see, Current Protocols in Molecular Biology, Vol. 2, Ed. Ausubel, et al., Greene Publish. Assoc. & Wiley Interscience, Ch. 13, 1988; Grant, et al., “Expression and Secretion Vectors for Yeast,” in Methods in Enzymology, Eds. Wu & Grossman, 1987, Acad. Press , N.Y., Vol. 153, pp. 516-544, 1987; Glover, DNA Cloning, Vol. II, IRL Press, Wash., D.C., Ch. 3, 1986; and Bitter, “Heterologous Gene Expression in Yeast,” Methods in Enzymology, Eds. Berger & Kimmel, Acad. Press, N.Y., Vol. 152, pp. 673-684, 1987; and The Molecular Biology of the Yeast Saccharomyces, Eds. Strathern et al., Cold Spring Harbor Press, Vols. I and II, 1982. A constitutive yeast promoter such as ADH or LEU2 or an inducible promoter such as GAL may be used (“Cloning in Yeast,” Ch. 3, R. Rothstein In: DNA Cloning Vol. 11, A Practical Approach, Ed. DM Glover, IRL Press, Wash., D.C., 1986). Alternatively, vectors may be used which promote integration of foreign DNA sequences into the yeast chromosome.

Nucleotide sequences of the invention may also be inserted into an expression system which expresses an agent which modulates expression or activity of synGAP or synGAP fusion or synGAP functional fragments thereof in an insect system (e.g., Drosophilia). In one such system, Autographa californica nuclear polyhedrosis virus (AcNPV) is used as a vector to express foreign or mutated polynucleotide sequences. The virus grows in Spodoptera frugiperda cells. The sequence encoding a protein of the invention may be cloned into non-essential regions (for example, the polyhedrin gene) of the virus and placed under control of an AcNPV promoter (for example the polyhedrin promoter). Successful insertion of the sequences coding for a protein of the invention will result in inactivation of the polyhedrin gene and production of non-occluded recombinant virus (i.e., virus lacking the proteinaceous coat coded for by the polyhedrin gene). These recombinant viruses are then used to infect S. frugiperda cells in which the inserted gene is expressed, see Smith, et al., J. Viol. 46:584, 1983; Smith, U.S. Pat. No. 4,215,051.

The vectors of the invention can be used to transform a host cell. As used herein, the term “transform” or “transformation” or equivalents thereof, refers to a permanent or transient genetic change induced in a cell following incorporation of new DNA (i.e., DNA exogenous to the cell). Where the cell is a mammalian cell, a permanent genetic change is generally achieved by introduction of the DNA into the genome of the cell.

As used herein, a “transformed cell” or “host cell” generally refers to a cell (e.g., prokaryotic or eukaryotic) into which (or into an ancestor of which) has been introduced, by means of recombinant DNA techniques, a DNA molecule encoding an agent which modulates expression or activity of synGAP or functional fragment thereof.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, methods of transfection or transformation with DNA include calcium phosphate co-precipitates, conventional mechanical procedures such as microinjection, electroporation, insertion of a plasmid encased in liposomes, or virus vectors, as well as others known in the art, may be used. Eukaryotic cells can also be co-transfected with DNA sequences encoding an agent which modulates expression or activity of synGAP or synGAP fusion or synGAP functional fragments thereof and a second foreign DNA molecule encoding synGAP, or a selectable marker, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). Typically, a eukaryotic host will be utilized as the host cell. The eukaryotic cell may be a yeast cell (e.g., Saccharomyces cerevisiae), an insect cell (e.g., Drosophila sp.) or may be a mammalian cell, including a human cell.

Eukaryotic systems, and mammalian expression systems, allow for post-translational modifications of expressed mammalian proteins to occur. Eukaryotic cells which possess the cellular machinery for processing of the primary transcript, glycosylation, phosphorylation, and, advantageously secretion of the gene product should be used. Such host cell lines may include, but are not limited to, CHO, VERO, BHK, HeLa, COS, MDCK, Jurkat, HEK-293, and WI38.

Mammalian cell systems which utilize recombinant viruses or viral elements to direct expression may be engineered. For example, when using adenovirus expression vectors, a polynucleotide encoding an agent which modulates expression or activity of synGAP, or synGAP fusion, or synGAP functional fragments thereof may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a encoding an agent which modulates expression or activity of synGAP, or synGAP fusion, or synGAP functional fragments thereof in infected hosts (e.g., see Logan & Shenk, Proc. Natl. Acad. Sci. USA, 81:3655-3659, 1984). Alternatively, the vaccinia virus 7.5K promoter may be used. (e.g., see, Mackett, et al., Proc. Natl. Acad. Sci. USA, 79:7415-7419, 1982; Mackett, et al., J. Virol. 49:857-864, 1984; Panicali, et al., Proc. Natl. Acad. Sci. USA 79:4927-4931, 1982). Of particular interest are vectors based on bovine papilloma virus which have the ability to replicate as extra chromosomal elements (Sarver, et al., Mol. Cell. Biol. 1:486, 1981). Shortly after entry of this DNA into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression a gene encoding an agent which modulates expression or activity of synGAP or synGAP fusion or synGAP functional fragments thereof gene in host cells. High level expression may also be achieved using inducible promoters, including, but not limited to, the metallothionine IIA promoter and heat shock promoters.

For long-term, high-yield production of recombinant proteins, stable expression may be required. Rather than using expression vectors which contain viral origins of replication, host cells can be transformed with the cDNA encoding an agent which modulates expression or activity of synGAP, or synGAP fusion, or synGAP functional fragments thereof controlled by appropriate expression control elements (e.g., promoter, enhancer, sequences, transcription terminators, polyadenylation sites, and the like), and a selectable marker. The selectable marker in the recombinant vector confers resistance to the selection and allows cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective media. A number of selection systems may be used, including, but not limited to, the herpes simplex virus thymidine kinase (Wigler, et al., Cell, 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy, et al., Cell, 22:817, 1980) genes can be employed in tk-, hgprt- or aprt-cells respectively. Also, anti-metabolite resistance can be used as the basis of selection for dhfr, which confers resistance to methotrexate (Wigler, et al., Proc. Natl. Acad. Sci. USA, 77:3567, 1980; O'Hare, et al., Proc. Natl. Acad. Sci. USA, 8:1527, 1981); gpt, which confers resistance to mycophenolic acid (Mulligan & Berg, Proc. Natl. Acad. Sci. USA, 78:2072, 1981; neo, which confers resistance to the aminoglycoside G-418 (Colberre-Garapin, et al., J. Mol. Biol. 150:1, 1981); and hygro, which confers resistance to hygromycin (Santerre, et al., Gene 30:147, 1984) genes. Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA 85:8047, 1988); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory, ed., 1987).

In order to amplify agents of the invention that are nucleic acid molecules, methods of amplifying such molecules are standard and well known in the art, including those that use of nucleic acid primers and the like. As used herein, the term “primer” refers to an oligonucleotide, whether natural or synthetic, which is capable of acting as a point of initiation of synthesis when placed under conditions in which primer extension is initiated or possible. Synthesis of a primer extension product which is complementary to a nucleic acid strand is initiated in the presence of nucleoside triphosphates and a polymerase in an appropriate buffer at a suitable temperature. For instance, if a nucleic acid sequence is inferred from a protein sequence, a primer generated to synthesize nucleic acid sequence encoding the protein sequence is actually a collection of primer oligonucleotides containing sequences representing all possible codon variations based on the degeneracy of the genetic code. One or more of the primers in this collection will be homologous with the end of the target sequence. Likewise, if a “conserved” region shows significant levels of polymorphism in a population, mixtures of primers can be prepared that will amplify adjacent sequences.

In another embodiment, the invention provides a method for identifying an agent and/or screening for agents, in vitro or in vivo, which modulate the expression or activity of synGAP, synGAP fusion, or functional fragments thereof. In this embodiment, a cell, subject, or sample is contacted with an agent suspected or known to have synGAP polypeptide expression modulating activity. The change in synGAP polypeptide gene expression is then measured as compared to a control or standard sample (e.g., in a Northern or Western blot). The control or standard sample can be the baseline expression of the cell or subject prior to contact with the agent.

Double-stranded interfering RNA molecules (as discussed above) are especially useful to inhibit expression of a target gene. For example, double-stranded RNA molecules can be injected into a target cell or organism to inhibit expression of a gene and the resultant gene products activity. It has been found that such double-stranded RNA molecules are more effective at inhibiting expression than either RNA strand alone. (Fire et al., Nature, 1998, 19:391(6669):806-11).

Methods of using an oligonucleotide to stall transcription are also contemplated, and is known as the triplex strategy since the oligomer winds around double-helical DNA, forming a three-strand helix. Therefore, these triplex compounds can be designed to recognize a unique site on a chosen gene (Maher, et al., Antisense Res. and Dev., 1:227, 1991; Helene, Anticancer Drug Design, 6:569, 1991).

Methods of targeted delivery is also encompassed by the invention. For example, one method targeted delivery is recombinant expression vectors such as a chimeric virus or a colloidal dispersion system or by injection. Various viral vectors which can be utilized for gene therapy as taught herein include adenovirus, herpes virus, vaccinia, or, an RNA virus such as a retrovirus. The retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting a polynucleotide sequence of interest into the viral vector, along with another gene which encodes the ligand for a receptor on a specific target cell, for example, the vector is now target specific. Retroviral vectors can be made target specific by inserting, for example, a polynucleotide encoding a sugar, a glycolipid, or a protein. Targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing, for example, an antisense polynucleotide.

Another targeted delivery system for polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes has been classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. In general, the compounds bound to the surface of the targeted delivery system will be ligands and receptors which will allow the targeted delivery system to find and “home in” on the desired cells. A ligand may be any compound of interest which will bind to another compound, such as a receptor.

Compositions of the invention can be administered parenterally (e.g., by injection or by gradual perfusion over time), enterically, by injection (e.g., intravenously, intraperitoneally, intramuscularly, subcutaneously, intracavity, or transdermall), rapid infusion, nasopharyngeal absorption, dermal absorption, rectally and orally. As used herein, the term “administering” is accomplished by any means known to the skilled artisan. Pharmaceutically acceptable carrier preparations for parenteral administration include sterile or aqueous or non-aqueous solutions, suspensions, and emulsions. Examples of non-aqueous solvents are propylene glycol, polyethylene glycol, vegetable oils such as olive oil, and injectable organic esters such as ethyl oleate. Carriers for occlusive dressings can be used to increase skin permeability and enhance antigen absorption. Liquid dosage forms for oral administration may generally comprise a liposome solution containing the liquid dosage form. Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners and elixirs containing inert diluents commonly used in the art, such as purified water.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents and inert gases and the like.

It is envisioned that the invention can be used to treat pathologies associated with nervous tissue, neurological disorders, or nervous insult (e.g., neurodegenerative diseases and associated diseases). As used herein, the term “nervous tissue” refers to the various components that make up the nervous system including, without limitation, neurons, neural support cells, glia, Schwann cells, vasculature contained within and supplying these structures, the central nervous system, the brain, the brain stem, the spinal cord, the junction of the central nervous system with the peripheral nervous system, the peripheral nervous system, and allied structures.

Examples of neurological disorders that are treatable by the method of using the present invention include, without limitation, trigeminal neuralgia; glossopharyngeal neuralgia; Bell's Palsy; myasthenia gravis; muscular dystrophy; amyotrophic lateral sclerosis; progressive muscular atrophy; progressive bulbar inherited muscular atrophy; herniated, ruptured or prolapsed invertebrate disk syndromes; cervical spondylosis; plexus disorders; thoracic outlet destruction syndromes; peripheral neuropathies such as those caused by lead, dapsone, ticks, porphyria, or Guillain-Barre syndrome; Alzheimer's disease; Huntington's Disease and Parkinson's disease.

As used herein, the term “neurodegenerative diseases” includes Alzheimer's disease, Parkinson's disease and Huntington's disease. Also as used herein, the term “nervous insult” refers to any damage to nervous tissue and any disability or death resulting there from. The cause of nervous insult may be metabolic, toxic, neurotoxic, iatrogenic, thermal or chemical, and includes without limitation, ischemia, hypoxia, cerebrovascular accident, trauma, surgery, pressure, mass effect, hemorrhage, radiation, vasospasm, neurodegenerative disease, infection, Parkinson's disease, amyotrophic lateral sclerosis (ALS), myelination/demyelination process, epilepsy, cognitive disorder, glutamate abnormality and secondary effects thereof. Thus, the present invention encompasses methods for ameliorating a disorder associated with degenerative disorders.

Hence, the methods of the present invention are particularly useful for treating a neurological disorder selected from the group consisting of: peripheral neuropathy caused by physical injury or disease state; head trauma, such as traumatic brain injury; physical damage to the spinal cord; stroke associated with brain damage, such as vascular stroke associated with hypoxia and brain damage, focal cerebral ischemia, global cerebral ischemia, and cerebral reperfusion injury; demyelinating diseases, such as multiple sclerosis; and neurological disorders related to neurodegeneration, such as Alzheimer's Disease, Parkinson's Disease, Huntington's Disease and amyotrophic lateral sclerosis (ALS). Neural tissue damage resulting from ischemia and reperfusion injury and neurodegenerative diseases includes neurotoxicity, such as that seen in vascular stroke and global and focal ischemia.

Further, according to the invention, an effective therapeutic amount of the compounds and compositions described above are administered to animals to affect a neuronal activity, particularly one that is not mediated by NMDA neurotoxicity. Such neuronal activity may consist of stimulation of damaged neurons, promotion of neuronal regeneration, prevention of neurodegeneration and treatment of a neurological disorder. Accordingly, the present invention further relates to a method of effecting a neuronal activity in an animal, comprising administering an effective amount of the agent identified by the methods described to a subject in need thereof.

In another embodiment of the invention, methods are provided wherein the agent identified by the methods of the invention are “neuroprotective” or “preventing neurodegeneratiion.” As used herein, the term “neuroprotective,” refers to the effect of reducing, arresting or ameliorating nervous insult, and protecting, resuscitating, or reviving nervous tissue that has suffered nervous insult. Also as used herein, the term “preventing neurodegeneration” includes the ability to prevent neurodegeneration in patients diagnosed as having a neurodegenerative disease or who are at risk of developing a neurodegenerative disease. The term also encompasses preventing further neurodegeneration in patients who are already suffering from or have symptoms of a neurodegenerative disease.

Generally, the terms “treating”, “treatment” and the like are used herein to mean affecting a subject, tissue or cell to obtain a desired pharmacologic and/or physiologic effect. The effect may be prophylactic in terms of completely or partially preventing a disease or sign or symptom thereof, and/or may be therapeutic in terms of a partial or complete cure for an infection or disease and/or adverse effect attributable to the infection or disease. “Treating” as used herein covers any treatment of, or prevention of a disease in an invertebrate, a vertebrate, a mammal, particularly a human, and includes: (a) preventing the disorder from occurring in a subject that may be predisposed to the disorder, but has not yet been diagnosed as having it; (b) inhibiting the disorder, i.e., arresting its development; or (c) relieving or ameliorating the disorder, i.e., cause regression of the disorder. By “neurodegenerative disorder” is meant a disorder that is characterized as having an decrease in synGAP over normal levels. Such decreases in synGAP lead to degenerative diseases that include, for example, Alzheimer's Disease, Parkinson's disease, ALS, and the like.

In one embodiment of the invention, methods of ameliorating a pathologic disorder (e.g., nervous insult or neurological disorders discussed above) by administering an agent which modulates the expression or activity of synGAP (e.g., increasing or activating) polypeptide, a polypeptide or peptide derivative of the agent, mimetics of the agent, a drug of the agent, chemical or combination of chemicals or a synGAP polypeptide-modulating agent into a form suitable for administration to a subject using carriers, excipients and additives or auxiliaries. As used herein, the term “subject” refers to a mammal, for example, a human, but may be any organism. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

In one embodiment of the invention, there is provided a method for identifying an agent which interacts with or modulates expression or activity of a synGAP polypeptide including incubating components comprising an agent and a synGAP polypeptide, or a recombinant cell expressing a synGAP polypeptide, under conditions sufficient to allow the agent to interact and determining the affect of the agent on the expression or activity of the synGAP gene or polypeptide, respectively. The term “affect”, as used herein, encompasses any means by which gene expression or protein activity can be modulated, and includes measuring the interaction of the agent with the synGAP protein by physical means including, for example, fluorescence detection of the binding of a the protein to a substrate or binding agent. Such agents can include, for example, polypeptides, peptidomimetics, chemical compounds, small molecules (e.g., chemical) and biologic agents as described below.

Accordingly, methods of the invention include incubating the agent under conditions which allow contact between the test agent and a synGAP polypeptide, a cell expressing a synGAP polypeptide or nucleic acid encoding a synGAP polypeptide. Contacting includes in solution and in solid phase. The test agent may optionally be a combinatorial library for screening a plurality of agents. Agents identified in the method of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology, 3:1008-1012, 1985), oligonucleotide ligation assays (OLAs) (Landegren, et al., Science, 241:1077, 1988), and the like. Molecular techniques for DNA analysis have been reviewed (Landegren, et al., Science, 242:229-237, 1988). Thus, the methods of the invention includes combinatorial chemistry methods for identifying chemical agents that bind to or affect synGAP polypeptide expression or activity.

Areas of investigation are the development of therapeutic treatments. The screening identifies agents that provide modulation of synGAP polypeptide function in targeted organisms. Of particular interest are screening assays for agents that have a low toxicity or a reduced number of side effects for humans. In particular, since the invention provides for that synGAP is specifically found in neurons and its increased expression or activity results in the decrease or prevention of apoptosis, detection of the effect of the agent on apoptosis can be easily assayed and thus the identification of potential therapeutics is provided by the present invention.

In another embodiment, nucleic acid probes encoding the agent which modulates the expression or activity of synGAP can be used to identify the agent from a specimen obtained from a subject. Examples of species from which nucleic acid sequence encoding a synGAP polypeptide can be derived include human, primate, swine, porcine, feline, canine, equine, murine, cervine, caprine, lupine, leporidine and bovine species.

Oligonucleotide probes, which correspond to a part of the sequence encoding the agent/protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucl. Acid Res. 9:879, 1981).

In one embodiment of the invention, purified nucleic acid fragments containing intervening sequences or oligonucleotide sequences of 10-50 base pairs are radioactively labeled. The labeled preparations are used to probe nucleic acids from a specimen by the Southern hybridization technique. Nucleotide fragments from a specimen, before or after amplification, are separated into fragments of different molecular masses by gel electrophoresis and transferred to filters that bind nucleic acid. After exposure to the labeled probe, which will hybridize to nucleotide fragments containing target nucleic acid sequences, binding of the radioactive probe to target nucleic acid fragments is identified by autoradiography (see Genetic Engineering, 1, ed. Robert Williamson, Academic Press, (1981), 72-81). Alternatively, nucleic acid from the specimen can be bound directly to filters to which the radioactive probe selectively attaches by binding nucleic acids having the sequence of interest. Specific sequences and the degree of binding is quantitated by directly counting the radioactive emissions.

For the most part, the probe will be detectably labeled with an atom or inorganic radical, most commonly using radionucleotides, but also heavy metals can be used. Conveniently, a radioactive label may be employed. Radioactive labels include ³²P, ¹²⁵I, ³H, ¹⁴C, ¹¹¹In, ⁹⁹Tc, or the like. Any radioactive label may be employed which provides for an adequate signal and has sufficient half-life. Other labels include ligands, which can serve as a specific binding pair member for a labeled ligand, and the like. A wide variety of labels routinely employed in immunoassays can readily be employed in the present assay. The choice of the label will be governed by the effect of the label on the rate of hybridization and binding of the probe to a nucleotide sequence. It will be necessary that the label provide sufficient sensitivity to detect the amount of a nucleotide sequence available for hybridization.

The manner in which the label is bound to the probe will vary depending upon the nature of the label. For a radioactive label, a wide variety of techniques can be employed. Commonly employed is nick translation with an a ³²P-dNTP or terminal phosphate hydrolysis with alkaline phosphatase followed by labeling with radioactive ³²P employing ³²P-NTP and T4 polynucleotide kinase. Alternatively, nucleotides can be synthesized where one or more of the elements present are replaced with a radioactive isotope, e.g., hydrogen with tritium. If desired, complementary labeled strands can be used as probes to enhance the concentration of hybridized label.

Standard hybridization techniques for detecting a nucleic acid sequence are known in the art. The particular hybridization technique is not essential to the invention. Other hybridization techniques are described by Gall and Pardue, Proc. Natl. Acad. Sci. 63:378, 1969); and John, et al., Nature, 223:582, 1969). As improvements are made in hybridization techniques they can readily be applied in the method of the invention.

The amount of labeled probe present in the hybridization solution will vary widely, depending upon the nature of the label, the amount of the labeled probe that can reasonably bind to the filter, and the stringency of the hybridization. Generally, substantial excess over stoichiometric concentrations of the probe will be employed to enhance the rate of binding of the probe to the fixed target nucleic acid.

In another embodiment of the invention, there is provided a transgenic animal. As used herein, the term “transgenic” is used to describe an animal which includes exogenous genetic material within all of its cells. A “transgenic” animal can be produced by cross-breeding two chimeric animals which include exogenous genetic material within cells used in reproduction. Twenty-five percent of the resulting offspring will be transgenic i.e., animals which include the exogenous genetic material within all of their cells in both alleles, 50% of the resulting animals will include the exogenous genetic material within one allele and 25% will include no exogenous genetic material.

The transgenic animals described herein are the result of a complete functional disruption (or knock-outs, ko) or incomplete functional disruption (conditional knock-outs, cond-ko) of the polynucleotide encoding synGAP. The terms “functional disruption” or “functionally disrupted” as used herein means that a gene locus comprises at least one mutation or structural alteration such that the functionally disrupted gene is incapable of directing the efficient expression of functional gene product. Knockout animals contain the same, artificially introduced mutation in every cell, abolishing the activity of a preselected gene. The resulting mutant phenotype (appearance, biochemical characteristics, behavior etc.) providing some indication of the gene's normal role in the non-human animal, and by extrapolation, in human beings. Knockout mouse models, as used herein, are widely used to study human diseases caused by the loss of gene function. Thus, in traditional constitutive knockout animals the mutation is present throughout development and in all cells of the adult. Conversely, in conditional knockout mice, different genetic strategies are incorporated, one such strategy is described herein, which allows mutations to be induced at different stages of development or in selected cell types.

Besides the methods described herein and in Vasquez et al. (2004), other methods to make transgenic non-human animals of the invention can be employed. Generally speaking, three such methods may be employed. In one such method, an embryo at the pronuclear stage (a “one cell embryo”) is harvested from a female and the transgene is microinjected into the embryo, in which case the transgene will be chromosomally integrated into both the germ cells and somatic cells of the resulting mature animal. In another such method, embryonic stem cells are isolated and the transgene incorporated therein by electroporation, plasmid transfection or microinjection, followed by reintroduction of the stem cells into the embryo where they colonize and contribute to the germ line. Methods for microinjection of mammalian species is described in U.S. Pat. No. 4,873,191. In yet another such method, embryonic cells are infected with a retrovirus containing the transgene whereby the germ cells of the embryo have the transgene chromosomally integrated therein. When the animals to be made transgenic are avian, because avian fertilized ova generally go through cell division for the first twenty hours in the oviduct, microinjection into the pronucleus of the fertilized egg is problematic due to the inaccessibility of the pronucleus. Methods to make transgenic animals described generally above. For example, U.S. Pat. No. 5,162,215 describes retrovirus infection for avian species. If micro-injection is to be used with avian species, however, a published procedure by Love et al., (Biotechnology, 12, January 1994) can be utilized whereby the embryo is obtained from a sacrificed hen approximately two and one-half hours after the laying of the previous laid egg, the transgene is microinjected into the cytoplasm of the germinal disc and the embryo is cultured in a host shell until maturity. When the animals to be made transgenic are bovine or porcine, microinjection can be hampered by the opacity of the ova thereby making the nuclei difficult to identify by traditional differential interference-contrast microscopy. To overcome this problem, the ova can first be centrifuged to segregate the pronuclei for better visualization.

The “non-human animals” of the invention include bovine, porcine, ovine and avian animals (e.g., cow, pig, sheep, chicken). The “transgenic non-human animals” of the invention are produced by introducing “transgenes” into the germ line of the non-human animal. Embryonal target cells at various developmental stages can be used to introduce transgenes. Different methods are used depending on the stage of development of the embryonal target cell. The zygote is the best target for micro-injection. The use of zygotes as is target for gene transfer has a major advantage in that in most cases the injected DNA will be incorporated into the host gene before the first cleavage (Brinster et al., Proc. Natl. Acad. Sci. USA 82:4438-4442, 1985). As a consequence, all cells of the transgenic non-human animal will carry the incorporated transgene. This will in general also be reflected in the efficient transmission of the transgene to offspring of the founder since 50% of the germ cells will harbor the transgene.

For methods utilizing microinjection of the nucleic acid, the transgene is first digested and purified free from any vector DNA, e.g., by gel electrophoresis. The transgene can include an operatively associated promoter which interacts with cellular proteins involved in transcription, ultimately resulting in constitutive expression. Promoters useful in this regard include those from cytomegalovirus (CMV), Moloney leukemia virus (MLV), and herpes virus, as well as those from the genes encoding metallothionin, skeletal actin, P-enolpyruvate carboxylase (PEPCK), phosphoglycerate (PGK), DHFR, and thymidine kinase. Promoters for viral long terminal repeats (LTRs) such as Rous Sarcoma Virus can also be employed. When the animals to be made transgenic are avian, promoters include those for the chicken l-globin gene, chicken lysozyme gene, and avian leukosis virus. Constructs useful in plasmid transfection of embryonic stem cells will employ additional regulatory elements well known in the art such as enhancer elements to stimulate transcription, splice acceptors, termination and polyadenylation signals, and ribosome binding sites to permit translation.

Retroviral infection can also be used to introduce the transgene into a non-human animal, as described above. The developing non-human embryo can be cultured in vitro to the blastocyst stage. During this time, the blastomeres can be targets for retroviral infection (Jaenich, R., Proc. Natl. Acad Sci USA 73:1260-1264, 1976). Efficient infection of the blastomeres is obtained by enzymatic treatment to remove the zona pellucida (Hogan, et al. (1986) in Manipulating the Mouse Embryo, Cold Spring Harbor Laboratory Press, Cold Spring Harbor, N.Y.). The viral vector system used to introduce the transgene is typically a replication-defective retro virus carrying the transgene (Jahner, et al., Proc. Natl. Acad. Sci. USA 82: 6927-6931, 1985; Van der Putten, et al., Proc. Natl. Acad Sci USA 82: 6148-6152, 1985). Transfection is easily and efficiently obtained by culturing the blastomeres on a monolayer of virus-producing cells (Van der Putten, supra; Stewart, et al., EMBO J. 6: 383-388, 1987). Alternatively, infection can be performed at a later stage. Virus or virus-producing cells can be injected into the blastocoele (D. Jahner et al., Nature 298: 623-628, 1982). Most of the founders will be mosaic for the transgene since incorporation occurs only in a subset of the cells which formed the transgenic nonhuman animal. Further, the founder may contain various retro viral insertions of the transgene at different positions in the genome which generally will segregate in the offspring. In addition, it is also possible to introduce transgenes into the germ line, albeit with low efficiency, by intrauterine retroviral infection of the midgestation embryo (D. Jahner et al., supra).

Another type of target cell for transgene introduction is the embryonal stem cell (ES). ES cells are obtained from pre-implantation embryos cultured in vitro and fused with embryos (M. J. Evans et al., Nature 292:154-156, 1981; M. O. Bradley et al., Nature 309:255-258, 1984; Gossler, et al., Proc. Natl. Acad Sci. USA 83:9065-9069, 1986; and Robertson et al., Nature 322:445-448, 1986). Transgenes can be efficiently introduced into the ES cells by DNA transfection or by retro virus-mediated transduction. Such transformed ES cells can thereafter be combined with blastocysts from a nonhuman animal. The ES cells thereafter colonize the embryo and contribute to the germ line of the resulting chimeric animal. (For review see Jaenisch, R., Science 240:1468-1474, 1988).

As used herein, the term “transgene” means any piece of DNA which is inserted by artifice into a cell, and becomes part of the genome of the organism (i.e., either stably integrated or as a stable extra chromosomal element) which develops from that cell. Such a transgene may include a gene which is partly or entirely heterologous (i.e., foreign) to the transgenic organism, or may represent a gene homologous to an endogenous gene of the organism. Included within this definition is a transgene created by the providing of an RNA sequence which is transcribed into DNA and then incorporated into the genome. The transgenes of the invention include DNA sequences which encode synGAP or a polynucleotide sequence operably linked thereto. For example, a polynucleotide sequence encoding synGAP or a functional fragment thereof operably linked to a selectable marker flanked by regions of sequence having homology to synGAP, and include polynucleotides, which may be expressed in a transgenic non-human animal.

As used herein, the term “transgenic” also includes any organism whose genome has been altered by in vitro manipulation of the early embryo or fertilized egg or by any transgenic technology to induce a specific gene knockout. The term “gene knockout” as used herein, refers to the targeted disruption of a gene in vivo with complete loss of function that has been achieved by any transgenic technology familiar to those in the art. In one embodiment, transgenic animals having gene knockouts are those in which the target gene has been rendered nonfunctional by an insertion targeted to the gene to be rendered non-functional by homologous recombination. As used herein, the term “transgenic” includes any transgenic technology familiar to those in the art which can produce an organism carrying an introduced transgene or one in which an endogenous gene has been rendered non-functional or “knocked out (ko).”

After an embryo has been microinjected, colonized with transfected embryonic stem cells or infected with a retrovirus containing the transgene (except for practice of the subject invention in avian species which is addressed elsewhere herein) the embryo is implanted into the oviduct of a pseudopregnant female. The consequent progeny are tested for incorporation of the transgene by Southern blot analysis of blood or tissue samples using transgene specific probes. PCR is particularly useful in this regard. Positive progeny (G0) are crossbred to produce offspring (G1) which are analyzed for transgene expression by Northern blot analysis of tissue samples.

The animals contemplated for use in the practice of the subject invention include, rattus sp., avian sp. canine sp., non-human primate sp., feline sp., mouse sp. etc. For purposes of the subject invention, these animals are referred to as “transgenic” when such animal has had a heterologous DNA sequence, or one or more additional DNA sequences normally endogenous to the animal (collectively referred to herein as “transgenes”) chromosomally integrated into the germ cells of the animal. The transgenic animal (including its progeny) will also have the transgene fortuitously integrated into the chromosomes of somatic cells.

In one embodiment of the invention, there is a functional disruption of the endogenous synGAP gene, and is not capable of encoding a functional protein and is therefore a functionally disrupted synGAP gene locus. In addition, a targeted mutation in an exon of an endogenous synGAP gene may result in a mutated endogenous gene that can express a truncated synGAP protein that is non-functional. Functional disruption can include the complete substitution of a heterologous synGAP gene locus in place of an endogenous synGAP locus, so that, for example, a targeting transgene that replaces the entire mouse synGAP locus with a human synGAP allele, which may be functional in the mouse, is said to have functionally disrupted the endogenous murine synGAP locus by displacing it. At least one exon which is incorporated into the mRNAs encoding most or all of the synGAP isoforms are functionally disrupted. Deletion or interruption of essential transcriptional regulatory elements, polyadenylation signal(s), and splicing site sequences will also yield a functionally disrupted gene. Functional disruption of an endogenous synGAP gene, may also be produced by other methods (e.g., antisense polynucleotide gene suppression). The term “structurally disrupted” refers to a targeted gene wherein at least one structural sequence (e.g., an exon sequence) has been altered by homologous gene targeting (e.g., by insertion, deletion, point mutation(s), and/or rearrangement). Typically, synGAP alleles that are structurally disrupted are consequently functionally disrupted, however synGAP alleles may also be functionally disrupted without concomitantly being structurally disrupted, i.e., by targeted alteration of a non-exon sequence such as ablation of a promoter. An allele comprising a targeted alteration that interferes with the efficient expression of a functional gene product from the allele is referred to in the art as a “null allele” or “knockout allele”.

As used herein, the terms “isoform”, or “synGAP isoform” refer to a polypeptide that is encoded by at least one exon and includes a sequence as set forth in GenBank Accession No. AF048976 (Chen et al., 1998).

In some embodiments, the endogenous non-human synGAP alleles are functionally disrupted so that expression of endogenously encoded synGAP is suppressed or eliminated. In one variation, an endogenous synGAP allele is targeted for disruption by homologous recombination.

Another method of the invention is to interrupt essential structural and/or regulatory elements of an endogenous synGAP gene by targeted insertion of a polynucleotide sequence, and thereby functionally disrupt the endogenous synGAP gene. For example, a targeting construct can homologously recombine with an endogenous synGAP gene and insert a non-homologous sequence, such as a neo expression cassette, into a structural element (e.g., an exon) and/or regulatory element (e.g., enhancer, promoter, splice site, polyadenylation site) to yield a targeted synGAP allele having an insertional interruption. The inserted sequence can range in size from about 1 nucleotide (e.g., to produce a frameshift in an exon sequence) to several kilobases or more, as limited by efficiency of homologous gene targeting with targeting constructs having a long nonhomologous replacement region.

Targeting constructs of the invention can also be employed to replace a portion of an endogenous synGAP gene with an exogenous sequence (i.e., a portion of a targeting transgene); for example, an exon of a synGAP gene may be replaced with a substantially identical portion that contains a nonsense or missense mutation.

In one embodiment, inactivation of an endogenous murine synGAP locus is achieved by targeted disruption of the appropriate gene by homologous recombination in a mouse embryonic stem cell. For inactivation, any targeting construct that produces a genetic alteration in the target synGAP gene locus resulting in the prevention of effective expression of a functional gene product of that locus may be employed. If only regulatory elements are targeted, some low-level expression of the targeted gene may occur (i.e., the targeted allele is “leaky”), however the level of expression may be sufficiently low that the leaky targeted allele is functionally disrupted.

In one embodiment of the invention, an endogenous synGAP gene in a non-human host is functionally disrupted by homologous recombination with a targeting construct that does not comprise a functionally equivalent sequence. In this embodiment, a portion of the targeting construct integrates into an essential structural or regulatory element of the endogenous synGAP gene locus, thereby functionally disrupting it to generate a null allele. Typically, null alleles are produced by integrating a non-homologous sequence encoding a selectable marker (e.g., a neo gene expression cassette) into an essential structural and/or regulatory sequence of a synGAP gene by homologous recombination of the targeting construct homology regions with endogenous synGAP gene sequences, although other strategies may be employed.

Most usually, a targeting construct is transferred by electroporation or microinjection into a totipotent embryonal stem (ES) cell line, such as the murine AB-1 or CCE lines. The targeting construct homologously recombines with endogenous sequences in or flanking an synGAP gene locus and functionally disrupts at least one allele of the synGAP gene. Typically, homologous recombination of the targeting construct with endogenous synGAP locus sequences results in integration of a non-homologous sequence encoding a selectable marker, such as neo, usually in the form of a positive selection cassette. The functionally disrupted allele is termed an synGAP null allele. ES cells having at least one synGAP null allele are selected for by propagating the cells in a medium that permits the preferential propagation of cells expressing the selectable marker. Selected ES cells are examined by PCR analysis and/or Southern blot analysis to verify the presence of a correctly targeted synGAP allele. Breeding of non-human animals which are heterozygous for a null allele may be performed to produce non-human animals homozygous for said null allele (or “knockout” or ko animals; Donehower et al., Nature 256:215, 1992; incorporated herein by reference). In some instances, breeding animals to maintain heterozygosity may be desired. As described more fully below, the transgenic organisms of the invention have utility as both heterozygous and homozygous synGAP null alleles. Alternatively, ES cells homozygous for a null allele having an integrated selectable marker can be produced in culture by selection in a medium containing high levels of the selection agent (e.g., G418 or hygromycin). Heterozygosity and/or homozygosity for a correctly targeted null allele can be verified with PCR analysis and/or Southern blot analysis of DNA isolated from an aliquot of a selected ES cell clone and/or from tail biopsies.

If desired, a transgene encoding, for example, a heterologous synGAP polypeptide comprising any of the mutations described herein can be transferred into a non-human host having a synGAP null allele, into a non-human ES cell that is homozygous for the synGAP null allele. It is generally advantageous that the transgene comprises a promoter and enhancer which drive expression of structural sequences encoding a functional heterologous mutation synGAP gene product. Thus, for example, a knockout mouse homozygous for null alleles at the synGAP locus can serve as a host for a transgene which encodes and expresses a gene associated with a neurological disorder or disease, e.g., with an Alzheimer's Disease Associated phenotype.

Several gene targeting techniques have been described, including but not limited to: co-electroporation, single-crossover integration, and double-crossover recombination (Bradley et al., Bio/Technology 10:534, 1992). The invention can be practiced using essentially any applicable homologous gene targeting strategy known in the art. The configuration of a targeting construct depends upon the specific targeting technique chosen. For example, a targeting construct for single-crossover integration targeting need only have a single homology region linked to the targeting region, whereas a double-crossover replacement-type targeting construct requires two homology regions, one flanking each side of the replacement region.

Without wishing to be bound by any particular theory of homologous recombination or gene conversion, it is believed that in such a double-crossover replacement recombination, a first homologous recombination (e.g., strand exchange, strand pairing, strand scission, strand ligation) between a first targeting construct homology region and a first endogenous synGAP gene sequence is accompanied by a second homologous recombination between a second targeting construct homology region and a second endogenous synGAP gene sequence, thereby resulting in the portion of the targeting construct that was located between the two homology regions replacing the portion of the endogenous synGAP that was located between the first and second endogenous synGAP sequences. For this reason, homology regions are generally used in the same orientation (i.e., the upstream direction is the same for each homology region of a transgene to avoid rearrangements). Double-crossover replacement recombination thus can be used to delete a portion of an endogenous synGAP gene and concomitantly transfer a non-homologous portion (e.g., a neo gene expression cassette) into the corresponding chromosomal location. Double-crossover recombination can also be used to add a non-homologous portion into an endogenous synGAP gene without deleting endogenous chromosomal portions. However, double-crossover recombination can also be employed simply to delete a portion of an endogenous synGAP gene sequence without transferring a non-homologous portion into the endogenous synGAP gene. Upstream and/or downstream from the nonhomologous portion may be a gene which provides for identification of whether a double-crossover homologous recombination has occurred; that is, it may be used for negative selection.

The positive selectable marker encodes a selectable marker which affords a means for selecting cells which have integrated targeting transgene sequences. The negative selectable marker encodes a selectable marker which affords a means for selecting cells which do not have an integrated copy of the negative selection expression cassette. Thus, by a combination positive-negative selection protocol, it is possible to select cells that have undergone homologous replacement recombination and incorporated the portion of the transgene between the homology regions (i.e., the replacement region) into a chromosomal location by selecting for the presence of the positive marker and for the absence of the negative marker.

Selectable markers for inclusion in the targeting constructs of the invention encode and express a selectable drug resistance marker and/or a HSV thymidine kinase enzyme. Suitable drug resistance genes include, for example: gpt (xanthine-guanine phosphoribosyltransferase), which can be selected for with mycophenolic acid; neo (neomycin phosphotransferase), which can be selected for with G418 or hygromycin; and DFHR (dihydrofolate reductase), which can be selected for with methotrexate (Mulligan and Berg (1981) Proc. Natl. Acad. Sci. U.S.A. 78: 2072; southern and Berg (1982) J. Mol. Appl. Genet. 1: 327; which are incorporated herein by reference).

Selection for correctly targeted recombinants will generally employ at least positive selection, wherein a non-homologous expression cassette encodes and expresses a functional protein (e.g., neo or gpt) that confers a selectable phenotype to targeted cells harboring the endogenously integrated sequence, so that, by addition of a selection agent (e.g., G418 or mycophenolic acid) such targeted cells have a growth or survival advantage over cells which do not have an integrated sequence.

Selection for correctly targeted homologous recombinants also employ negative selection, so that cells bearing only non-homologous integration of the transgene are selected against. Typically, such negative selection techniques employ an expression cassette encoding the herpes simplex virus thymidine kinase gene (HSV tk) positioned in the transgene so that it integrates only by non-homologous recombination. Such positioning generally as accomplished by linking the HSV tk expression cassette (or other negative selection marker) distal to the recombinant homology regions so that double-crossover replacement recombination of the homology regions transfers the positive selection expression cassette to a chromosomal location but does not transfer the HSV tk gene (or other negative selection marker) to a chromosomal location. A nucleoside analog, gancyclovir, which is preferentially toxic to cells expressing HSV tk, can be used as the negative selection agent, as it selects for cells which do not have an integrated HSV tk expression marker. FIAU may also be used as a selective agent to select for cells lacking HSV tk.

Generally, targeting constructs of the invention include: (1) a positive selection marker flanked by two homology regions that are substantially identical to host cell endogenous synGAP gene sequences, and (2) a distal negative selection marker. However, targeting constructs which include only a positive selection marker can also be used. Typically, a targeting construct will contain a positive selection marker, which includes a neo gene linked downstream (i.e., towards the carboxyl-terminus of the encoded polypeptide in translational reading frame orientation) of a promoter such as the HSV tk promoter or the pgk promoter.

Targeting constructs of the invention have homology regions that are highly homologous to the predetermined target endogenous DNA sequencers), for example, isogenic (i.e., identical sequence). Isogenic or nearly isogenic sequences may be obtained by genomic cloning or high-fidelity PCR amplification of genomic DNA from the strain of non-human animals which are the source of the ES cells used in the gene targeting procedure.

In another aspect of the invention, conditional or controllable transgenic animals, as described in WO99/31969 (incorporated herein in its entirety by reference) are also encompassed by this invention. In such animals the inserted gene is under the control of a regulatable promoter or other expression control system.

Conditional knockout mice are created using the Cre/Lox system. “LoxP” or “lox” refers to a short (34 bp) DNA sequence that is recognized by Cre recombinase of the E. coli bacteriophage P1. Placement of two loxP sites in the same orientation on either side of a DNA segment will result, in the presence of Cre recombinase, in efficient excision of the intervening DNA segment, leaving behind only a single copy of the loxP site. Conditional knockouts are created by introducing the Cre gene into the ES cell under the control of a regulatable promoter of another expression control system.

Peptides to other phosphorylated and unphosphorylated sequences near phosphorylated serine residues are within the scope of the invention, for example, any serines residues which are potentially phosphorylated as described herein, or any which are later discovered are included.

For making transgenic non-human animals (which include homologously targeted non-human animals), embryonal stem cells (ES cells) can be used. The embryonic stem cells described herein can be obtained and manipulated according to published procedures (Teratocarcinomas and Embryonic Stem Cells: A Practical Approach, E. J. Robertson, ed., IRL Press, Washington, D.C. (1987); Zjilstra et al., Nature 342:435-438 (1989); and Schwartzberg et al., Science 246:799-803 (1989), each of which is incorporated herein by reference). Murine ES cells, such as AB-1 line grown on mitotically inactive SNL76/7 cell feeder layers (McMahon and Bradley (1990) Cell 62: 1073) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al. (1987) Nature 326: 292-295), the D3 line (Doetschman et al. (1985) J. Embryol. Exp. Morph. 37: 27-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the gertn cells of the resulting animal). The blastocysts containing the injected ES cells are allowed to develop in the uteri of pseudopregnant nonhuman females and are born as chimeric mice. The resultant transgenic mice are chimeric for cells having inactivated endogenous synGAP loci and are backcrossed and screened for the presence of the correctly targeted transgene(s) by PCR or Southern blot analysis on tail biopsy DNA of offspring so as to identify transgenic mice heterozygous for the inactivated synGAP locus. By performing the appropriate crosses, it is possible to produce a transgenic non-human animal homozygous for functionally disrupted synGAP alleles. Such transgenic animals are substantially incapable of making an endogenous synGAP gene product.

Non-human animals comprising transgenes which are heterozygous null or homozygous null for synGAP can be used commercially as controls or standards in the development of neurodegenerative therapeutics and diagnostics. For example, it is contemplated that the synGAP-knockout organisms of the invention can be used as controls in screens for agents which modulate expression or activity of synGAP. Such agents can be developed as pharmaceuticals for treating neurodegenerative conditions, e.g., Alzheimer's disease, Parkinson's disease, ALS and the like. Other uses include using cells (particularly neuronal cells) derived from the synGAP-knockout organisms for creating protein expression profiles between synGAP-knockout organisms and organisms of identical species having a phenotype associated with Alzheimer's Disease.

The pharmaceutical compositions comprising substantially the agent which modulates the expression or activity of synGAP are suitable for systemic administration to the host, including both parenteral, topical, and oral administration. The pharmaceutical compositions may be administered parenterally, e.g., subcutaneously, intramuscularly, or intravenously. Thus, the present invention provides compositions for administration to a host, where the compositions comprise a pharmaceutically acceptable solution of the identified compound in an acceptable carrier, as described above.

Transgenic organisms and/or effects the agents on organisms (e.g., organisms having a phenotype associated with a neurodegenerative disease) can be screened for presence of the transgene or changes in disease phenotypes in several ways. For example, in subjects with Alzheimer's disease, RNA expression can be detected and analyzed and the copy number and/or level of expression are determined using methods known to those of skill in the art. The transgenic animals or organisms displaying a phenotype associated with Alzheimer's disease can also be observed for clinical changes. Examples of neurobehavioral disorders for evaluation are poor mating response, agitation, diminished exploratory behavior in a novel setting, inactivity, seizures and premature death.

Brain regions known to be affected by the disorder (e.g., CA, cerebellum, cortex and the like) of interest are particularly reviewed for changes. When the disease of interest is Alzheimer's disease, the regions reviewed include the cortico-limbic region, including APP excretions, gliosis, changes in glucose uptake and utilization and plaque formation. However, in strains of animals which are not long-lived, not all behavioral and/or pathological changes associated with a particular disease may be observed. Comparing any of the foregoing with a synGAP-knockout organisms can provide useful information in identifying novel therapeutic agents and diagnostics.

The transgenic organisms (e.g., synGAP knockout organisms) of the invention can be used as controls for tester organisms for agents of interest, e.g. antioxidants such as Vitamin E or lazaroids, thought to confer protection against the development of Alzheimer's disease. A test organism is treated with the agent of interest, and the neuropathology or behavioral pathology is compared to the synGAP-knockout organisms of the invention, wherein a neuropathology or behavior in the test animal treated with the agent of interest that is substantially similar to or superior to that of the synGAP-knockout organisms is an indication of protection from AD. The indices used are those which can be detected in a live animal, such as changes in performance on learning and memory tests. The effectiveness can be confirmed by effects on pathological changes when the animal dies or is sacrificed.

In another embodiment of the invention, agents which modulate the expression or activity of synGAP also affect the signaling complex associated with NMDA receptors. NMDA or glutamate receptors play a role in numerous neurological, neurodegenerative, psychiatric, and psychological disorders, and a variety of mammalian disease states are associated with aberrant activity of these receptors. Glutamate receptors have been classified as either “ionotropic” or “metabotropic”. Ionotropic receptors are directly coupled to the opening of cation channels in the cell membranes of the neuron. Metabotropic receptors belong to the family of G-protein-coupled receptors and are coupled to systems that lead to enhanced phosphoinositide hydrolysis, activation of phospholipase D, increases or decreases in cAMP formation, and changes in ion channel function.

Metabotropic glutamate receptors (mGluRs) are divided into three groups based on amino acid sequence homology, transduction mechanism and binding selectivity: Group I, Group II and Group III. Group I includes metabotropic glutamate receptors 1 and 5 (mGluR1 and mGluR5), Group II includes metabotropic glutamate receptors 2 and 3 (mGluR2 and mGluR3), and Group III includes metabotropic glutamate receptors 4, 6, 7, and 8 (mGluR4, mGluR6, mGluR7 and mGluR8). Each mGluR type may be found in several subtypes. For example, subtypes of mGluR1 include mGluR1a, mGluR1b and mGluR1c.

At excitatory postsynaptic terminals of glutamatergic neurons, calcium influx through the NMDA-type glutamate receptor (NMDA receptor) contributes to the control of a wide variety of neuronal processes, including synaptic plasticity, synapse formation, and cell death. A core complex of signaling proteins associated with the NMDA receptor is found in the postsynaptic density (PSD) fraction (Kennedy, 1997; 2000; Sheng & Sala, 2001), and is postulated to relay signals controlled by activation of the NMDA receptor. Among the most prominent proteins in this complex are PSD-95, Ca²⁺/calmodulin-dependent protein kinase II (CaMKII) and synGAP (Chen et al., 1998; Kim et al., 1998), a Ras GAP protein also recently shown to possess Rap GAP activity (Krapivinsky et al., 2004). In the PSD, synGAP associates with the PSD-95 family and MUPP1 scaffold proteins (Chen et al., 1998; Kim et al., 1998; Zhang et al., 1999; Krapivinsky et al., 2004) and is phosphorylated by CaMKII, which increases its GAP activity 2-fold (Oh et al., 2004), potentially decreasing the amount of activated Ras at the synapse. Through this mechanism, synGAP may integrate NMDA receptor activation and Ras signaling pathways and thus influence many neuronal processes

RasGAP's have previously been implicated in regulation of developmental programs in the brain. Neurons from mice lacking synGAP undergo precocious synapse formation (Vazquez et al., 2004). Mice lacking the related protein, p120-RasGAP, show a variety of developmental defects including extensive embryonic neuronal cell death (Henkemeyer et al., 1995). These effects may be due, in part, to deregulation of Ras. Ras-dependent pathways have been shown to promote both neuronal cell survival (Kurada & White, 1998; Bonni et al., 1999; Mazzoni et al., 1999; Botella et al., 2003) and cell death (Henkemeyer et al., 1995; Lin et al., 1998; Lee et al., 1999), indicating that a delicate balance of Ras activity is essential for the survival of neurons. The potential role of synGAP in activity-dependent regulation of Ras at synapses may be crucial for regulating the action of growth factors, refinement of synaptic connection, and neuronal cell death.

Parts of the invention are also described in Oh, S. J. et al., (Apr. 23, 2004) J. Biol. Chem. 279(17): 17980-17988, the entire contents of which is incorporated herein by reference.

The present invention is more particularly described in the following examples which are intended as illustrative only since numerous modifications and variations therein will be apparent to those skilled in the art. The following examples are intended to illustrate but not limit the invention.

### EXAMPLE 1

### SynGAP is Phosphorylated by Calcium-Calmodulin Kinase II

### Materials and Methods

The reagents and manufacture of the same used in the invention are as follows: Acetonitrile, UV/HPLC grade, was purchased from EM Science (Gibbstown, N.J.); HPLC/Spectra grade trifluoroacetic acid from Pierce (Rockford, Ill.); Iodoacetamide from Sigma (St. Louis, Mo.); [γ-32P]ATP and [α-32P]GTP from ICN Pharmaceuticals Inc. (Irvine, Calif.); modified sequencing grade trypsin from Promega (Madison, Wis.); C18 reverse phase HPLC columns (4.6×250 mm) from Vydac (Hesperia, Calif.); cellulose-coated TLC sheets (20×20 mm) from EM Science; glutathione-agarose from Sigma; and phosphorimager screens and scanner from Molecular Dynamics (Sunnyvale, Calif.). Calmodulin was purchased from Calbiochem (San Diego, Calif.). CaMKII was purified from rat forebrain as previously described (Miller et al. 1985).

Preparation of Postsynaptic Density Fraction from Rat Brain

The crude PSD fraction was prepared as described previously (Cho et al. 1992) by a modification of the method of Carlin et al. (Carlin et al. 1980). Expression and Purification of Glutathione-S-Transferase Fusion Proteins Containing Portions of synGAP-A vector for expression of a fusion protein containing a portion of the carboxyl tail of synGAP (residues 946 to 1167) fused to the carboxyl terminus of glutathione S-transferase (GST-ctSynGAP) was constructed in the pGEX plasmid, according to manufacturers instructions (Pharmacia Biotech Inc., Piscataway, N.J.). The fusion protein contained the sequence of synGAP encoded by positions 2836 to 3501 in the synGAP cDNA (Accession No. AF048976). E. coli cells transformed with the plasmids were grown in cultures containing 50 μg/ml ampicillin at 37° C. At mid-log phase, expression was induced by addition of 0.1 mM isopropyl-thiogalactoside and continued until late log phase. Cells were harvested and frozen at −80° C. Cells were lysed by sonication in 137 mM NaCl, 2.7 mM KCl, 4.3 mM Na2HPO4, 1.4 mM KH2PO4, pH 7.3, (PBS) containing 1% Triton X-100, proteinase inhibitor cocktail (Roche Pharmaceuticals, Mannheim, Germany), 0.1 mM PMSF, 0.5 mM dithiothreitol (DTT). After centrifugation at 15,000×g for 10 min, lysate supernates were incubated at room temperature (RT) for 2 hrs, or at 4° C. overnight with glutathione-agarose beads. In some cases, to recover more fusion protein, the pellets were also resuspended in PBS plus 1% n-lauryl sarcosine, 1% Triton X-100, and 0.1 mM PMSF. The suspension was sonicated and subjected to centrifugation at 15,000×g. The supernate from this spin was pooled with the lysate supernates and incubated with glutathione-agarose beads for 2 hrs at 4° C. The bead suspension was transferred to a column and the beads were washed with PBS. GST-fusion proteins were eluted in 50 mM Tris (pH 8.0), 20 mM reduced glutathione, and 0.1 mM PMSF at 4° C. The protein concentration was determined by a modification of the method of Lowry (Petersen 1983), and stored at −80° C.

### Expression of SynGAP in Insect Cells

The entire sequence encoding synGAP was inserted into plasmid pVL1392 (Pharmingen; San Diego, Calif.) at EcoR1 and BamH1 restriction sites, which added a FLAG tag to the amino terminus. The recombinant FLAG-tagged synGAP (rSynGAP) was expressed in Hi-5 insect cells by the Caltech Protein Expression Laboratory. Cells were harvested by centrifugation, and the cell pellets were frozen at −80° C. Pellets were resuspended in 20 mM Tris-Cl (pH 8.0), 2 mM EDTA, 2 mM DTT, 0.1 mM PMSF, 0.5% Triton X-100, 1 μg/ml Deoxyribonuclease I, and Roche protease inhibitor cocktail, and lysed by homogenization at 4° C. Nuclei were removed by centrifugation at 100 g and membranes were harvested at 100,000 g. Like endogenous synGAP, rSynGAP is tightly bound to membranes. All attempts to extract it resulted in loss of GAP activity. Membrane fractions from control insect cells in which synGAP was not present had no detectable GAP activity (data not shown). Thus, a measure of synGAP activity in the membrane fractions was observed. Recombinant synGAP in the membranes was detected by immunoblotting with an anti-FLAG antibody (Sigma).

Phosphorylation of synGAP and GST-ctSynGAP by CaMKII

Phosphorylation by purified CaMKII was carried out in a reaction mix containing 50 mM Tris-HCl (pH 8.0), 10 mM MgCl2, 0.7 mM CaCl2, 0.4 mM EGTA, 30 μM [γ-32P]ATP (1000-3000 cpm/pmol) or 30 μM ATP, 10 μg/ml calmodulin, 10 mM DTT, 3 μg purified rat brain CaMKII and Hi-5 cell membranes containing 1.5 to 2 μg rSynGAP, or 45 ng purified CaMKII and 3 μg GST-ctSynGAP. Phosphorylation was initiated by addition of CaMKII and ATP to a 30 μl reaction mix prewarmed to 30° C. for 2 min. The reaction was carried out for 2 min, or as indicated, and stopped by the addition of SDS-PAGE sample buffer. The mixture was placed in a boiling water bath for 3 min, then samples were fractionated by SDS-PAGE on 10% gels for GST-ctSynGAP, and 7.5% gels for rSynGAP. The gels were dried, and exposed to X-Ray film to identify phosphorylated proteins. To quantify the amount of phosphate incorporated, the level of 32P in the bands was determined with a STORM PhosphorImager (Molecular Dynamics). The relative densities measured by the Imager were converted to cpm by comparison to signals from standard amounts of 32P-phosphate spotted onto filter paper and imaged at the same time.

Determination of Stoichiometry of Phosphorylation of synGAP by CaMKII

The moles of synGAP per mg protein in either the postsynaptic density or the membranes of Hi-5 insect cells was determined from immunoblots containing increasing amounts of protein from each sample, labeled with affinity purified primary antibodies specific for synGAP (Affinity Bioreagents, Golden, Colo.), and secondary antibodies conjugated to Alexa fluor-488 (Molecular Probes, Eugene, Oreg.). The labeling was quantified with the use of the STORM system, and compared with the labeling of standard amounts of GST-ctSynGAP protein, which contains the epitopes of synGAP used to prepare the primary antibody. The nmoles phosphate incorporated into the synGAP band (determined as described above) per nmoles of total synGAP was then calculated.

### Trypsinization of Phosphorylated GST

Fusion Proteins and Recombinant SynGAP—Proteins were phosphorylated as described above with the following modifications. The reaction was conducted in a volume of 500 μl containing 80-120 μg/ml of GST-fusion protein or 300-400 μg/ml of Hi-5 membrane protein containing recombinant synGAP. The reaction mixture was preincubated at 30° C. for 5 min, and the reaction was initiated by addition of 2-4 μg/ml of CaMKII for phosphorylation of GST-fusion protein or 10-20 μg/ml of CaMKII for phosphorylation of membrane-bound recombinant synGAP in the presence of 30 μM [γ-32P]ATP (1500-3000 cpm/pmol). The reaction was continued for 5, 15, or 30 min at 30° C. and stopped by addition of 3×SDS sample buffer. The mixture was placed in a boiling water bath for 3 min, then fractionated by SDS-PAGE. The gel was stained with Coomassie blue R-250 and the appropriate band of GST-fusion protein or recombinant synGAP was identified by comparison to molecular weight markers. Bands were excised, chopped into small pieces, and transferred to 1.5 ml Eppendorf tubes. The pieces were incubated for 30 min at 37° C. in 2 mM tris-(2-carboxyethyl) phosphine hydrochloride (TCEP), 50% acetonitrile, 0.5 M ammonium bicarbonate (pH>8) to reduce the protein and destain the gel piece. The protein was then alkylated by transferring the gel piece to 25 mM iodoacetamide, 50% acetonitrile, 25 mM ammonium bicarbonate and incubating at RT in the dark for 20 min. Gel pieces were rinsed with 50 mM ammonium bicarbonate. Proteins were then trypsinized in the gel as previously described (Hellman et al. 1995).

### HPLC Fractionation of Phosphopeptides

Trypsinized phosphopeptides were fractionated by HPLC on a C18 reverse phase column developed at 1 mL/min with a gradient of 0-30% acetonitrile in 0.1% trifluoroacetic acid. Radioactivity in each 0.5 mL fraction was measured in a Beckman LS 7800 scintillation counter by detection of Cerenkov radiation.

### Mass Spectrometry and Sequencing of Phosphopeptides

Mass spectrometry was conducted by the Protein and Peptide Microanalytical Laboratory at Caltech with a PerSeptive Biosystems/Vestec Lasertech II reflector for matrix-assisted, laser desorption ionization, time-of-flight (MALDI-TOF) mass spectrometry. Data were collected in both linear and reflector modes. Serine phosphopeptides are reliably identified by appearance of a new peptide in reflector mode that is reduced in mass by about 98 amu from the parent, because of cleavage of a phosphate group from the parent. The identity of some of the phosphopeptides detected herein was confirmed by amino acid sequencing by Tandem mass spectrometry (MS/MS).

Identification of Tryptic Peptides with Peptidesort Software

The Peptidesort software package (GCG, Accyleris, Inc.) permits identification of a peptide from its molecular mass and its relative retention time during HPLC. This was the program used to predict all possible tryptic peptides, sorted by retention time and molecular mass, from the amino acid sequence of synGAP. The retention times from HPLC and the molecular masses of each phosphopeptide detected by mass spectrometry with those predicted by the program was compared. Mass spectrometry measures the mass of the peptide fragment plus a proton; whereas, the program predicts the mass of the corresponding hydrolysis product. Therefore, for comparison, 17 amu was subtracted from the mass of each peptide predicted by the program. After this correction, unless noted, the differences between masses predicted by the program, and those of peptides determined by mass spectrometry and reported in Tables I and II were less than about 1 amu.

### Assay of Ras GTPase-Activating Activity

GAP assays of 20-35 μg of PSD and 5-15 μg of recombinant synGAP were performed after phosphorylation as described above, in the absence or presence of 0.3 mM free Ca2+ and 10 μg/ml calmodulin for 2 min at 30° C., but in a final volume of 30 μl. In some reactions, antibody 6G6 against PSD-95 or inhibiting antibodies 4A11 and 6E9 (Molloy et al. 1991) against CaMKII (20 μg each of IgG partially purified from Ascites fluid by precipitation with 50% ammonium sulfate) were included in the reaction. The phosphorylation reactions were stopped by addition of 60 μl of a mixture to bring the solution to 0.66 mM EGTA, 11.7 μM okadaic acid, 22 mM Tris-Cl (pH 7.5), 9.3 mM MgCl2, 111 mM NaCl, and 2.2 mM DTT. The GAP assay was then initiated by addition of 10 μl containing 2 pmol [α-32P]GTP-bound GST-Ras fusion protein (Chen et al. 1998) for a total volume of 100 μl GAP reaction mixture (Bollog et al. 1995). The reactions were carried out at 30° C. for 15 min, stopped by addition of 600 μL of ice-cold 5% glutathione-agarose beads and 80 μl of ice-cold 50 mM EDTA, and then incubated at 4° C. for 45 min on an end-over-end mixer. Beads were washed three times (Smith et al. 1995), nucleotides were dissociated from the column-bound GST-Ras, and [α-32P]GTP and [α-32P]GDP was separated by thin layer chromatography (Bollog et al. 1995). The separated nucleotides were visualized by PhosphorImager analysis (Molecular Dynamics) and quantified with Image Quant software to determine the percent GDP generated in the assay (GDP/[GDP+GTP]×100), a measure of Ras GTPase activity.

### Site-Directed Mutagenesis of Recombinant SynGAP

Various mutant constructs were prepared to study the effect of mutation of the identified phosphorylation sites to alanine (serines 764, 765, 1058, 1062, 1064, 1093, 1095, 1123). Mutagenic oligonucleotides (18-25mer) that contained alanine instead of the identified serine were synthesized at the Caltech Oligonucleotide Synthesis Laboratory. The oligonucleotides were phosphorylated at the 5′ end by T4 kinase, then annealed to the denatured synGAP plasmid (pVL1392) at room temperature for 30 minutes. The oligonucleotides were extended with T4 DNA polymerase and T4 DNA ligase in vitro to generate a hemi-methylated, double-stranded DNA molecule. A restriction digestion was performed with Dpn-1 to eliminate non-mutant plasmid DNA (those with two methylated strands). The DNA molecules were then transformed into the E. coli mutS strain (deficient in the methylation-specific repair system) and colonies were screened by DNA sequencing for plasmids containing the alanine mutations (Kramer et al. 1984). Plasmids containing the desired mutations were transformed into E. coli DH5α for propagation, and mutations were confirmed by DNA sequencing.

### Production of Phosphosite-Specific Antibodies

Synthetic peptides with the sequence ITKQH-S(PO3)-QTPSTC (P-S1123) and RGLNS-S(PO3)-MDMARC (P-S765) were purchased from SynPep (Dublin, Calif.). Purified peptides were conjugated via succinimidyl 4-(N-maleimidomethyl)cyclohexane-1-carboxylate to keyhole limpet hemocyanin. Rabbit antisera against the conjugated P-S1123 and P-S765 were raised by CoCalico Biologicals (Reamstown, Pa.) and Sigma Genosis (The Woodlands, Tex.), respectively. Specificity and optimum dilution was determined for each bleed by immunoblotting against 15-20 μg of PSD fraction phosphorylated by endogenous CaMKII, and of equivalent nonphosphorylated PSD fraction. Antibodies from sera that contained a high titer specific for phospho-synGAP were purified by peptide affinity chromatography.

Five mgs of P-S1123 or PS765 peptide were conjugated to 5 mL of Sulfolink coupling resin (Pierce, Rockford, Ill.) according to the manufacturer's instructions. The coupled resin was then mixed for one hour with a blocker consisting of 0.1 M cysteine in TE85 (50 mM Tris pH 8.5, 5 mM EDTA), then washed twice with 5 vol each of TE85, TE85, 1 M NaCl, and finally with 50 mM Tris pH 7.5, 5 mM EDTA (TE75). Before each use, the resin was blocked for 1 hr with 3 vol TE75, 20% non-immune rabbit serum, washed with TE75, G elution buffer (Pierce), and finally re-equilibrated with TE75 buffer. IgG from about 25 ml of serum was concentrated by precipitation in 50% ammonium sulfate, 0.1 M Tris-Cl, pH 7.5. The pellet was redissolved in 50 ml of TE75 and dialyzed against two changes of the same buffer. The dialyzed protein was stirred with the peptide resin for two hours, poured into a column and washed with 10 column volumes of TE75. Bound IgG was eluted in Gentle Elution Buffer (Pierce), collecting 1 ml fractions into tubes containing 0.1 ml of 1 M Tris-Cl pH 7.5. Protein was detected in each fraction by absorbance at 280 nm. Fractions containing high amounts of protein were further characterized by immunoblotting against both phosphorylated and nonphosphorylated PSD protein. Fractions with the highest concentration of phosphosite-specific antibodies were pooled.

### Preparation of Cortical Neuronal Cultures

Cultures of cortical neurons with less than 1% astrocytes were prepared from fetal mice (15-16 days gestation) as previously described (Rose et al. 1993) in 24 well plates coated with 50 ng/ml poly-D-lysine (Sigma) and 2 ng/ml laminin (BD Biosciences) and Neurobasal medium (Gibco), B27 supplement (Gibco), 0.5 mM Glutamax I (Gibco), 25 μM glutamate and 25 μM β-mercaptoethanol. After 3 days in vitro (DIV), Cytosine arabinoside (Sigma) was added (10 μM) to halt the growth of non-neuronal cells. Cells were used at 13-14 DIV.

### Cell Treatment and Protein Extraction

Cell cultures (13-14 days in vitro [DIV]) were washed 3 times in HEPES-control salt solution (HCSS) containing (in mM): 120 NaCl, 5.4 KCl, 0.8 MgCl2, 1.8 CaCl2, 10 NaOH, 20 HEPES, 5.5 glucose, pH 7.4, and then half were exposed to 25 μM N-methyl-D-aspartic acid (NMDA) dissolved in HCSS or to an equal amount of additional HCSS for 15 seconds (2-4 wells for each condition). After treatment, cells were washed quickly with ice-cold PBS and extracted with lysis buffer (1% SDS, 20 mM Tris-Cl pH 7.5, 10 mM EGTA, 40 mM β-glycerophosphate, 2.5 mM MgCl2, 2 mM orthovanadate, and complete mini protease inhibitor cocktail [Roche]). Extracts were heated at 90° C. for 5 min and insoluble material was removed by centrifugation at 14,000 g for 30 min. Protein concentrations were determined by the bicinchoninic acid method (Pierce) using bovine serum albumin as standard.

### Western Blotting

To determine synGAP phosphorylation, 5 μg of protein samples were dissolved in SDS-PAGE sample buffer, heated at 90° C. for 5 min., fractionated by SDS-PAGE on 8% gels and transferred to nitrocellulose membranes (Schleicher & Schuell) in transfer buffer containing 50 mM Tris, 380 mM Glycine, 0.1% SDS and 20% Methanol. Membranes were blocked with 5% milk in TBS-T buffer (20 mM Tris, 150 mM NaCl, 0.05% Tween 20), and were then incubated with phosphorylation site-specific antibodies anti-p-synGAP-1123 (1:5,000), anti-p-synGAP-765 (1:10,000) or antibody recognizing total synGAP (1:3,000). Bound antibodies were detected by the enhanced chemiluminescence method (Pierce).

### Results

### Stoichiometry and Rate of Phosphorylation of SynGAP by CaMKII

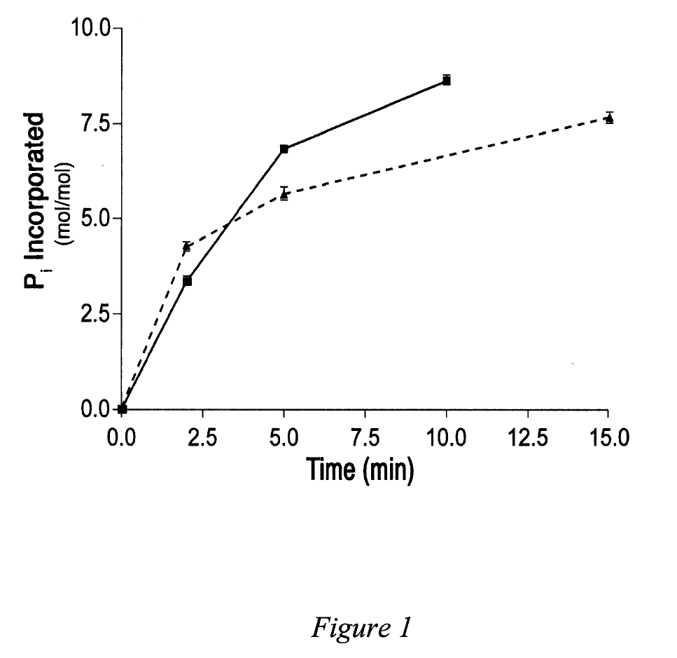

SynGAP in the postsynaptic density and recombinant synGAP are phosphorylated rapidly and to a high stoichiometry by CaMKII (FIG. 1). Reactions were carried out in the presence of [γ-32P]-ATP (1000 cpm/pmol), Ca2+ and calmodulin, as described above. At the indicated times, reactions were stopped by addition of SDS-PAGE sample buffer. Radio-labeled synGAP was fractionated by SDS-PAGE on 7.5% gels and visualized with a PhosphorImager. [γ-P32]-PO4 in the synGAP protein band was quantified with the use of ImageQuant software from Molecular Dynamics, as described under “Experimental Procedures”. Removal of synGAP from the PSD fraction or from Hi-5 cell membranes without destroying its GAP activity proved difficult, so experiments with synGAP in the PSD fraction phosphorylated by endogenous CaMKII, and with recombinant synGAP in Hi-5 cell membranes phosphorylated by purified rat forebrain CaMKII, was used. The most rapid phosphorylation occurs within about 2 min after which synGAP contains approximately 3 to 3.5 mol of phosphate/mol.

The Ras-GTPase Activating Activity of SynGAP is Increased after Phosphorylation by CaMKII

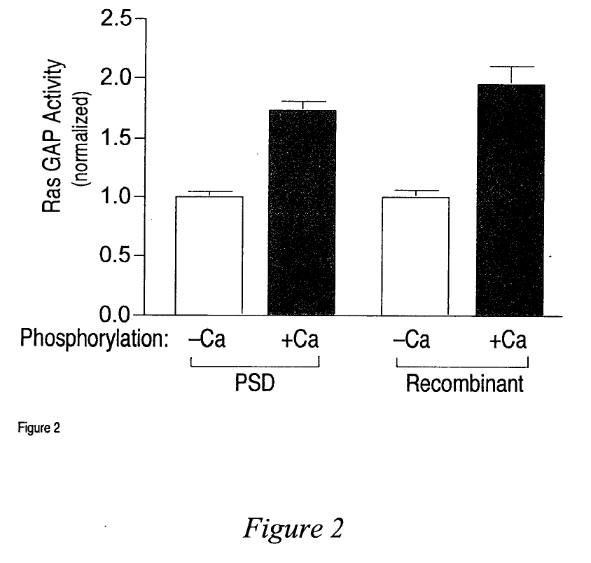

It was previously reported that the Ras GAP activity of synGAP is inhibited about 2-fold after phosphorylation by CaMKII (Chen et al. 1998). It was shown that this observation was caused by inhibition of GAP activity by ATP and pyrophosphate added to inhibit endogenous protein phosphatases (Oh et al. 2002). Hence, regulation of GAP activity of synGAP by CaMKII, after optimizing conditions for the GAP assay in the absence of pyrophosphate, was re-investigated as described above. To preserve the phosphorylation state of synGAP during the GAP assay (15 min), EGTA and 3.5 μM okadaic acid was added at the end of the phosphorylation reaction. These agents fully inhibited dephosphorylation of synGAP and did not interfere with the GAP assay (data not shown). Conditions under which the GAP activity is linear with time and with amount of synGAP. Under these conditions, phosphorylation by CaMKII increased the GAP activity of synGAP in the PSD fraction by about 75% and of recombinant synGAP by about 95% (FIG. 2).

FIG. 2 shows that synGAP in the PSD fraction (15 μg total PSD protein) was phosphorylated by endogenous CaMKII. Recombinant synGAP (15 μg of Hi-5 cell membrane protein) was phosphorylated by 3 μg added purified CaMKII. Phosphorylation of synGAP was carried out in the absence (−Ca) or presence (+Ca) of 0.7 mM CaCl₂ and 0.6 μM calmodulin (10 μg/ml) for 2 min at 30° C. Data are mean values from 4 independent experiments. GAP activities are plotted after subtraction of intrinsic Ras GTPase activity and are normalized to the activity of nonphosphorylated synGAP. Non-normalized GAP activity, before phosphorylation and after subtraction of the intrinsic Ras GTPase activity, was 11.6% hydrolysis of GTP for synGAP in the PSD fraction, and 19% for recombinant synGAP. The mean intrinsic Ras GTPase activity was 6.0% hydrolysis of GTP.

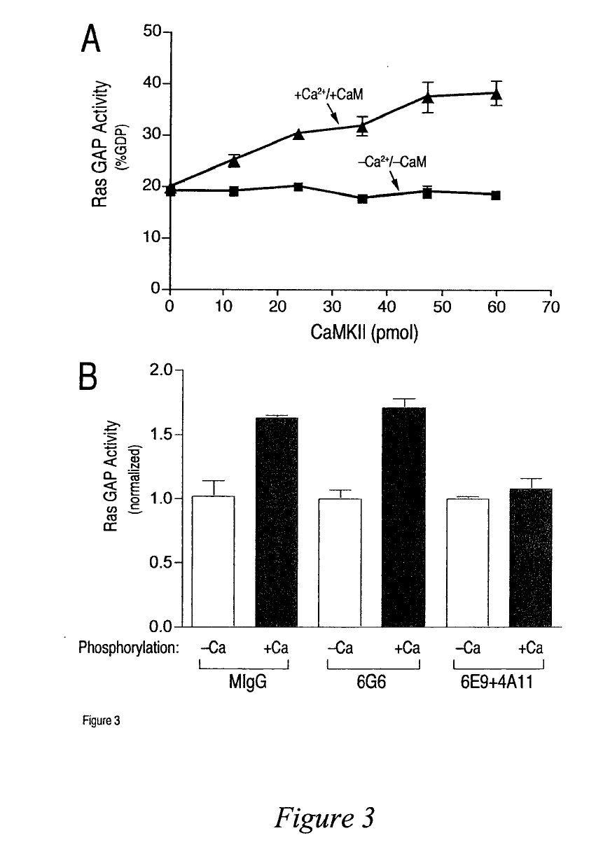

To verify that phosphorylation by CaMKII is responsible for the increase in activity, the dependence of the increase on the amount of exogenous CaMKII in the phosphorylation reaction was determined (FIG. 3A). FIG. 3A shows that recombinant synGAP (15 ug) was phosphorylated for 2 min at 30° C. in the presence of the indicated amounts of purified forebrain CaMKII, then assayed for GAP activity as described above. When no CaMKII was added to the assay, GAP activity was not increased. The importance of phosphorylation by CaMKII for the increase in GAP activity was further supported by an experiment in which inhibiting antibodies against CaMKII to the phosphorylation reaction was added (FIG. 3B). For example, monoclonal antibodies 6E9 and 4A11, which inhibit CaMKII activity, were incubated with the PSD fraction (15 ug each) at 4° C. for 30 minutes and then phosphorylation was carried out at 30° C. in the presence of CaCl2 and calmodulin as described above. In two control reactions, either mouse IgG or an anti-PSD-95 antibody (6G6) were substituted for inhibiting antibodies. The data are mean values of 3 independent experiments. GAP activities are plotted after subtraction of the intrinsic Ras GTPase activity and are normalized to the activity of nonphosphorylated synGAP. Non-normalized GAP activity, before phosphorylation and after subtraction of intrinsic Ras GTPase activity, was 14.5% hydrolysis of GTP in the presence of mouse IgG (MigG), 12.5% in the presence of 6G6, and 15.2% in the presence of 6E9+4A11. The mean intrinsic Ras GTPase activity was 6.8% hydrolysis of GTP. Thus, the increase in GAP activity in the PSD fraction was decreased when antibodies that inhibit CaMKII (4A11 & 6E9) were included in the phosphorylation reaction, but was unaffected by addition of non-immune mouse IgG or an antibody against PSD-95 (6G6).

### Identification of Phosphorylation Sites in the Carboxyl Terminal Portion of SynGAP

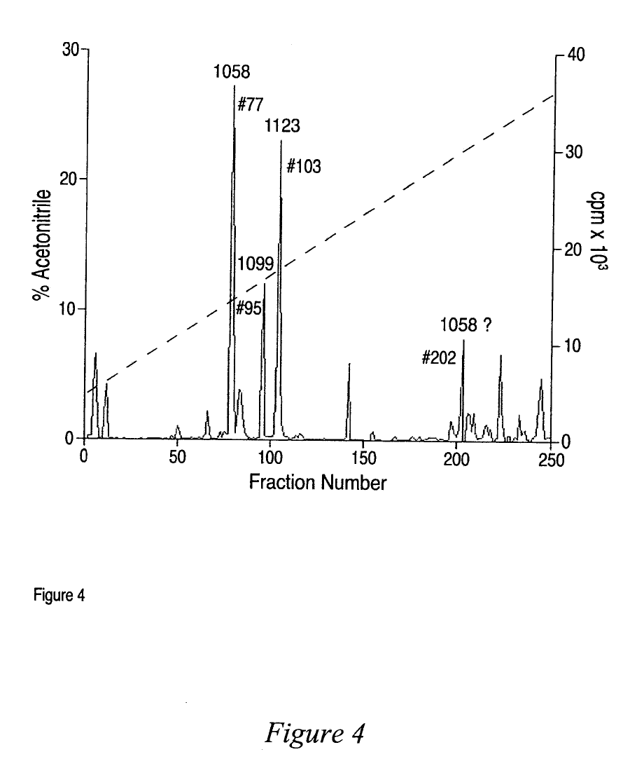

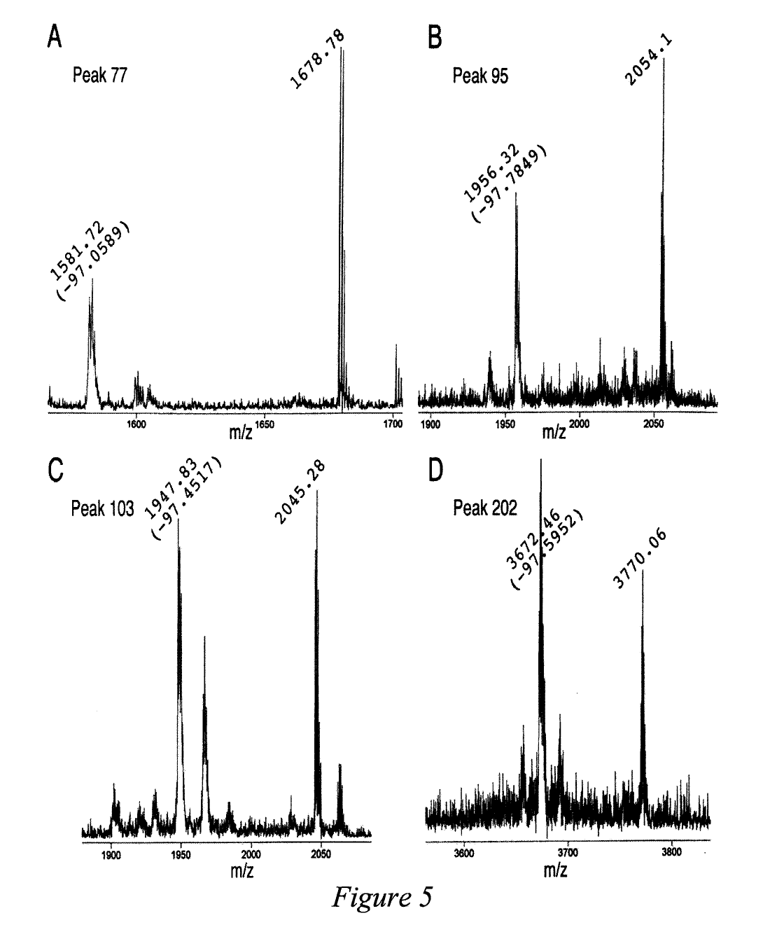

Various fusion proteins containing portions of synGAP were constructed, expressed, and then tested for phosphorylation by CaMKII as described above. The fusion protein containing the carboxyl terminal fragment of synGAP (aa 946-aa 1167) was rapidly phosphorylated (data not shown), therefore its phosphorylation sites were identified. Tryptic peptides were generated from the phosphorylated protein and fractionated by HPLC as described above. Purified GST-ctSynGAP fusion protein (60 μg), containing residues 946-1167 of synGAP, was phosphorylated for 30 minutes in the presence of [γ32P]-ATP at 30° C. The phosphorylated protein was fractionated by SDS-PAGE and digested in the gel with trypsin as described above. Tryptic peptides were eluted and fractionated by HPLC and the indicated radio labeled tryptic peptides were identified by mass spectrometry as described above (Table I and FIG. 5). Peaks were labeled with the fraction number (#) and the location of phosphorylated sites in full length synGAP corresponding to the phosphopeptide in the peak. Fractionation of trypsinized fusion protein after 5 and 15 minutes of phosphorylation produced peaks with the same retention times. The result shows four major peaks of radioactivity appeared reproducibly (FIG. 4).

The molecular masses of phosphopeptides shown in FIG. 4 were determined by MALDI-TOF mass spectrometry (FIG. 5). The identity of the phosphopeptide in fraction 202 is ambiguous because two peptides, a partial tryptic product containing site S1058 and a tryptic peptide containing 7 serines and 3 threonines, have masses slightly more than 1 amu different from the determined mass.

TABLE I
Identification of Phosphopeptides
in HPLC fractions shown in FIG. 4

| | | Peptide mass | | Predicted | |
|---|---|---|---|---|---|
| HPLC | Identified | (MALDI-TOF) | | peptide | Residues in |
| fractions | sites | Linear | Reflected** | mass*** | synGAP |
| 77* | S1058 | 1678.78 | 1581.72 | 1580.87 | 1056-1070 |
| 95* | S1099 | 2054.10 | 1956.32 | 1957.10 | 1097-1120 |
| 103* | S1123 | 2045.28 | 1947.83 | 1948.10 | 1121-1138 |
| 202 | S1058 | 3770.06 | 3672.46 | 3674.06 | 1056-1090 |
| | or 7 S's, | | | or | or |
| | 3 T's | | | 3673.90 | 970-1004 |

*Identify of phosphopeptide was confirmed by sequencing by MS/MS as described under “Experimental Procedures.”
**Mass of peptide after neutral loss of phosphate in reflector mode.
***The mass of peptides predicted from tryptic hydrolysis in Peptidesort software were corrected by subtracting 17 amu's to match the M + H⁺ mass measured by MALDI/TOF.

The same four peaks were present after 5, 15, or 30 min of phosphorylation, with the size of the peaks proportional to the time of phosphorylation, indicating that all the peaks represent sites that are phosphorylated at approximately the same rate. Individual fractions containing the peaks were concentrated and subjected to MALDI-TOF mass spectrometry in both linear and reflector modes. In reflector mode, neutral loss of H3PO4 from serine and threonine phosphopeptides usually produces a new peptide peak with a mass 98 amu less than that of the phosphopeptide itself (Annan et al. 1996). The appearance of this peak is diagnostic for a phosphopeptide. Each of the four samples showed one such new peak in reflector mode (FIG. 5, Table I), permitting identification the mass of the phosphopeptide in each sample. FIG. 5 shows post source decay spectra of phosphopeptide peaks 77 (A), 95 (B), 103 (C), and 202 (D) as that shown in FIG. 4. The ordinates are detector counts. Each spectrum shows corrected masses (M+H⁺) of peptides run in reflector mode in a MALDI/TOF spectrometer. The larger mass is that of the phosphopeptide. The smaller mass is that of the peptide after neutral loss of phosphate (about 98).

Three of the four phosphopeptides were pure enough to sequence by MS/MS as described above. The sequences revealed that fraction 77, 95, and 103 contained phosphorylated sites corresponding to serines 1058, 1099 and 1123 in full-length synGAP, respectively (Table I). The masses of the predicted tryptic peptides calculated with the program Peptidesort corresponded to those determined by mass spectrometry, confirming the identities of these phosphopeptides. The recovery of the phosphopeptide in fraction 202 was too low for sequencing by MS/MS. Its predicted mass could correspond to one of two peptides; a peptide containing residues 970 to 1004 that contains 7 serines and 3 threonines, or a peptide containing residues 1056 to 1090 resulting from partial tryptic digestion of the region containing phosphorylation site serine 1058. The predicted masses of both of these peptides are slightly greater than 1 amu different from the mass determined by mass spectrometry (Table I).

Identification of Additional Phosphorylation Sites after Mutation of S1058 and S1123 in Full Length synGAP

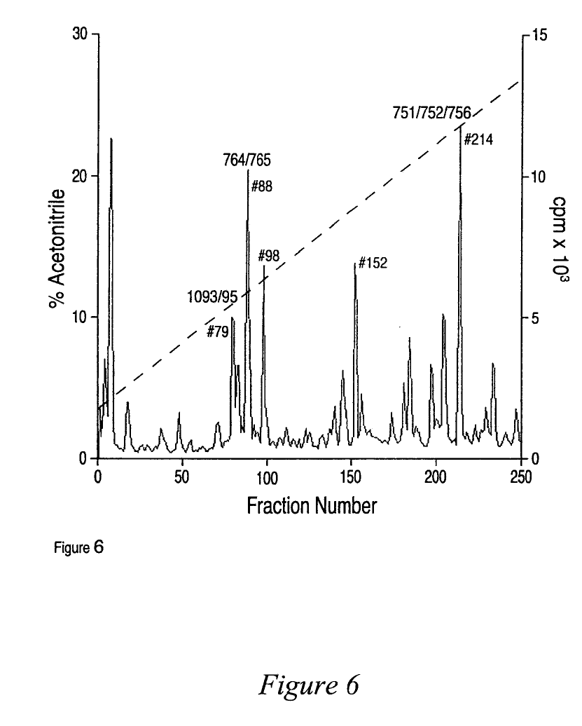

To check for additional phosphorylation sites in synGAP, a full length synGAP in which the two major phosphorylation sites, S1058 and S1123, were mutated to alanine (S1058/1123A) was constructed and expressed as described above. This mutant protein was still rapidly phosphorylated by purified CaMKII, but to a stoichiometry of only ˜1.5 mol PO4/mol synGAP compared to 3 to 3.5 for wild type synGAP (FIG. 1), indicating that the two mutated sites are indeed major sites phosphorylated rapidly in the native protein. However, the stoichiometry greater than one indicated that additional sites might be present in the portions of synGAP that were not contained in GST-ctSynGAP, or might be unmasked in the carboxyl terminal portion by mutation of the major sites. A tryptic peptide map of the phosphorylated S1058/1123A mutant, prepared as described in the previous section, revealed five major phosphopeptide peaks (FIG. 6).

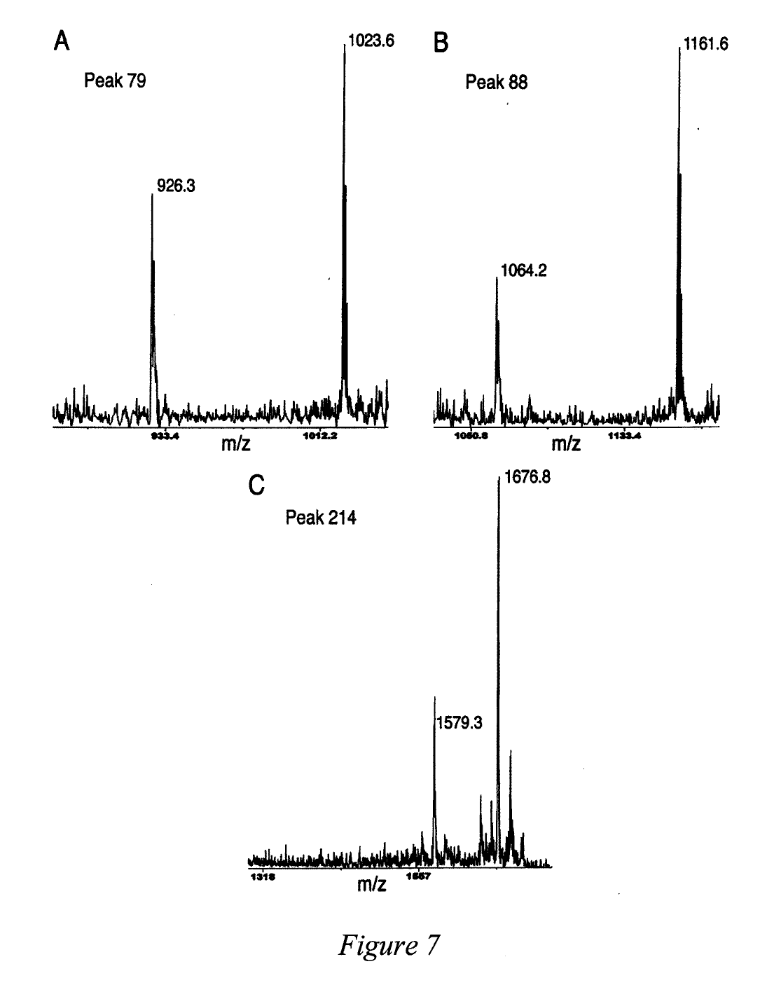

FIG. 6 shows that A cDNA encoding synGAP with two mutations, S1058A and S1123A (henceforth referred to as S1058/1123A), was expressed in Hi-5 cells as described under “Experimental Procedures”. The mutant protein (200 μg) was phosphorylated for 30 minutes at 30° C., as described above. The phosphorylated protein was fractionated by SDS-PAGE and digested in the gel with trypsin as described under “Experimental Procedures”. Tryptic peptides were eluted and fractionated by HPLC and the indicated radio labeled tryptic peptides were identified by mass spectrometry as described above (also see Table II and FIG. 7). Peaks were labeled with the fraction number (#) and the location of phosphorylated sites in full length synGAP corresponding to the phosphopeptide in the peak. Fractions 98 and 152 had detectable radioactivity, however no phosphopeptides were detected in them by MALDI-TOF mass spectrometry in the reflector mode.

The molecular masses of phosphopeptides shown in FIG. 6 were determined by MALDI-TOF mass spectrometry (FIG. 7). Phosphopeptides in fractions 79, 88, and 214 were identified by comparison with retention order and molecular masses predicted by the Peptidesort program, as described herein above.

TABLE II
Identification of phosphopeptides
in HPLC fractions shown in FIG. 6

| | | Peptide mass | | Predicted | |
|---|---|---|---|---|---|
| HPLC | Identified | (MALDI) | | peptide | Residues in |
| fractions | sites | Linear | Reflected* | mass** | synGAP |
| 79 | S1093/1095 | 1023.6 | 926.3 | 926.1 | 1089-1096 |
| 88 | S764/765 | 1161.6 | 1064.2 | 1064.2 | 761-770 |
| 214 | S750/ | 1676.8 | 1579.3 | 1579.7 | 747-760 |
| | 751/756 | | | | |

*Mass of peptide after neutral loss of phosphate in reflector mode.
**The mass of peptides predicted from tryptic hydrolysis in Peptidesort software were corrected by subtracting 17 amu's to match the M + H⁺ mass measured by MALDI/TOF.

MALDI-TOF mass spectrometry in linear and reflector modes, revealed the mass of phosphopeptides in fractions 79, 88 and 214 (FIG. 7 and Table II). FIG. 7 shows mass spectrographs of phosphopeptides from synGAP mutant missing sites 1058 and 1123. Post source decay spectra of phosphopeptide peaks 79 (A), 88 (B), and 214 (C) as shown in FIG. 6 are shown. The ordinates are detector counts. Each spectrum shows corrected masses (M+H+) of peptides run in reflector mode in a MALDI/TOF spectrometer. The larger mass is that of the phosphopeptide. The smaller mass is that of the peptide after neutral loss of phosphate (about 98).

Fraction numbers 98 and 152 contained several peptides, but none of them could be positively identified as a phosphopeptide (FIG. 6). The masses of phosphopeptides determined by mass spectrometry and their relative retention times during HPLC with those predicted in the program Peptidesort for specific tryptic peptides were matched (Table II). The phosphopeptide in fraction 79 is predicted to contain two serines at positions 1093 and 1095 in synGAP. The prominent phosphopeptide in fraction 88 is predicted to contain two adjacent serine residues at positions 764 and 765. The phosphopeptide in fraction 214 contains three serines at positions 750, 751, and 756. The two pairs of serines, 750/751 and 764/765 are located in two 13 residue tandem repeats that are 61% identical. These five potential sites between positions 750 and 765 are outside the region of the protein contained in GST-ctSynGAP and represent potential major sites of phosphorylation. In contrast, sites 1093 and 1095 were present in GST-ctSynGAP but were not significantly phosphorylated in the presence of Ser 1058 and Ser 1123 (FIG. 4), indicating that these sites may be minor sites in the wild type protein that are “unmasked” in the mutant. The phosphoprotein present in fraction 98 was unidentifiable, but its retention time is similar to that of the peak containing phosphorylation site 1099 in FIG. 4. Thus, serine at position 1099 may be significantly phosphorylated in both mutant and wild type synGAP.

The Major Phosphorylation Sites, Serines 764/765, 1058 and 1123 all Contribute to Regulation of GAP Activity of synGAP by CaMKII

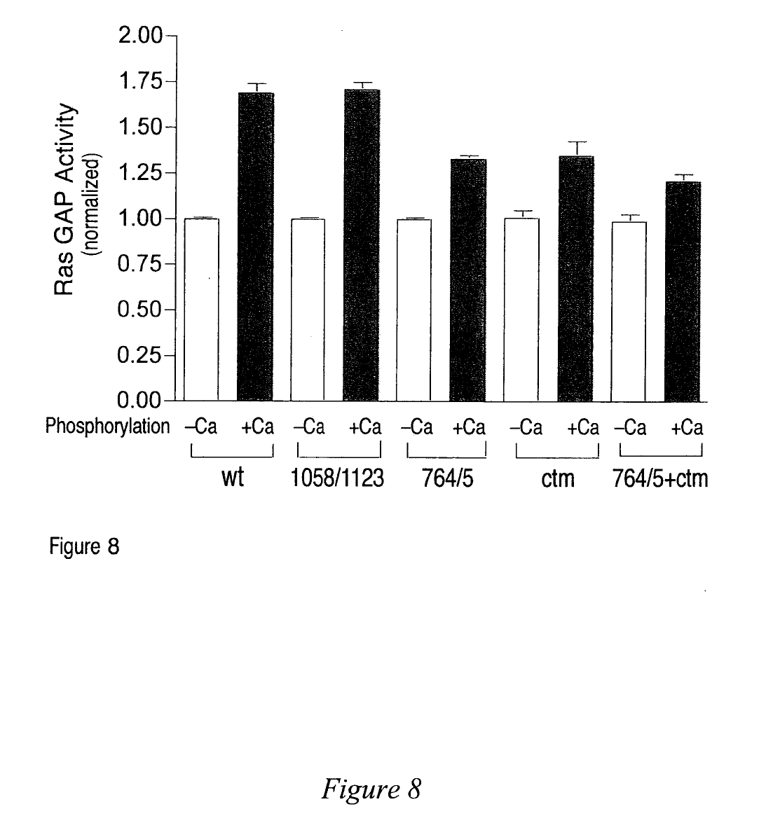

To determine which of the identified phosphorylation sites are responsible for regulation of synGAP's GAP activity, a series of synGAP mutants in which several of the phosphorylation sites, or combinations of them were mutated to alanine were generated. The GAP activity after phosphorylation of these mutants by CaMKII was then measured substantially as described above and shown in FIG. 2. The increase in GAP activity produced by phosphorylation was reduced from about 70% to about 40% in a mutant in which both serines 764 and 765 were changed to alanine (FIG. 8). The indicated synGAP mutants were generated by site-directed mutagenesis and expression in Hi-5 cells as described above. GAP assays were performed for 15 minutes after phosphorylation by CaMKII in the presence or absence of Ca2+/CaM for 2 min as described above. The data are mean values from 4 or more independent experiments. GAP activities are plotted after subtraction of intrinsic Ras GTPase activity and are normalized to the activity of nonphosphorylated synGAP. Non-normalized GAP activity, before phosphorylation and after subtraction of Ras GTPase, was 14% hydrolysis of GTP for wt synGAP; 11.8% for S1058/1123A; 14.1% for S764/5A; 17.2% for ctm (S1058A, S1062A, S1064A, S1093A, S1095A, and S1123A); and 17.4% for S764/5A+ctm. The mean intrinsic Ras GTPase activity was 6.5% hydrolysis of GTP.

In contrast, a mutant missing both serines 1058 and 1123 phosphorylation sites still showed nearly about a 70% increase in GAP activity after phosphorylation. Three of the other identified phosphorylation sites—1093, 1095, and 1099—are located near 1058 and 1123 and are phosphorylated in the mutant that is missing 1058 and 1123. It is possible that phosphorylation of those sites, or of other nearby serines such as 1062 and 1064, can mimic the effect of the phosphorylation of 1058 and 1123. Therefore, a carboxyl terminal synGAP mutant (ctm) in which six of the potential phosphorylation sites including, 1058, 1062, 1064, 1093, 1095, and 1123 are mutated to alanine was identified. The increase in GAP activity induced by phosphorylation of this mutant (termed the ctm mutant) was reduced to about 40%, similar to the increase observed for the 764/765 mutant (FIG. 8). The increase in GAP activity in a mutant in which all eight of these serine to alanine mutations are combined was determined. In this mutant, the increase in GAP activity after phosphorylation was reduced to about 20% (FIG. 8). Thus, it appears that these phosphorylation sites all can contribute to the regulation of GAP activity by CaMKII. It is possible that the remaining 20% increase in GAP activity results from mutation of phosphorylation of serines 750, 751, 756, or 1099.

### Activation of NMDA Receptors Increases Phosphorylation of SynGAP on Ser1123 and Ser765 in Cortical Neurons

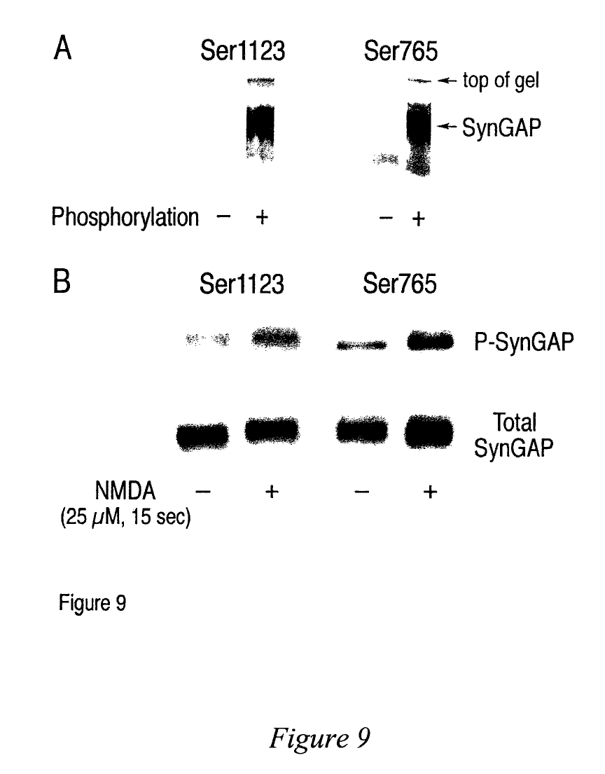

Phosphosite-specific antibodies against phosphorylated peptides containing the sequences surrounding ser765 and ser1123 were made substantially as described above. After affinity purification on columns substituted with the peptide antigens, these antibodies are highly specific for phosphorylated synGAP on immunoblots of the PSD fraction (FIG. 9A). Fractions were assayed by immunoblotting against purified postsynaptic density proteins before or after phosphorylation by CaMKII substantially as described above. Fractions with the greatest specificity for phospho-synGAP were pooled. Immunoblots of nonphosphorylated (−) and phosphorylated (+) postsynaptic density proteins were made with 1/20,000 dilutions of the pooled antibodies.

To determine whether Ser765 or Ser1123 can undergo phosphorylation in neurons, cortical cultures were exposed to 25 μM NMDA for 15 seconds, after which the cultures were extracted with SDS-PAGE sample solution. Immunoblots of extracts from control and treated cultures were prepared and probed with the antibodies against phosphosites, and with antibodies against synGAP to control for sample loading. As shown in FIG. 9B, treatment of the cultures with NMDA resulted in an increase in phosphorylation of synGAP on both sites, indicating that these sites can be phosphorylated in vivo following activation of NMDA receptors. For FIG. 9B, cultures were immediately dissolved in SDS-PAGE sample solution, boiled, fractionated by gel electrophoresis, and immunoblotted with affinity purified antibodies prepared against phosphopeptides with the sequence surrounding serine 765 or serine 1123 in synGAP, substantially as described above. Similar results were obtained in two experiments.

SynGAP is a Ras GTPase activating protein that was originally discovered as an abundant PSD protein that associates with the NMDA-receptor scaffold protein PSD-95 (Chen et al. 1998; Kim et al. 1998). It is a prominent component of a signaling complex that associates with the cytosolic face of the NMDA receptor at postsynaptic spines of excitatory synapses in the central nervous system and regulates a wide variety of synaptic functions in the developing and adult nervous systems (Kennedy 2000; Sheng et al., 2000). Mouse mutants in which synGAP is eliminated by homologous recombination die in the first or second week after birth, indicating that synGAP is essential for viability after birth (Komiyama et al. 2002; Kim et al. 2003). SynGAP is phosphorylated by the Ca2+-regulated protein kinase CaMKII, which is one of the major targets of Ca2+ flowing through activated NMDA receptors. To better understand how synGAP may participate in synaptic regulation through the NMDA-receptor, characterizing how synGAP is modified by CaMKII was established.

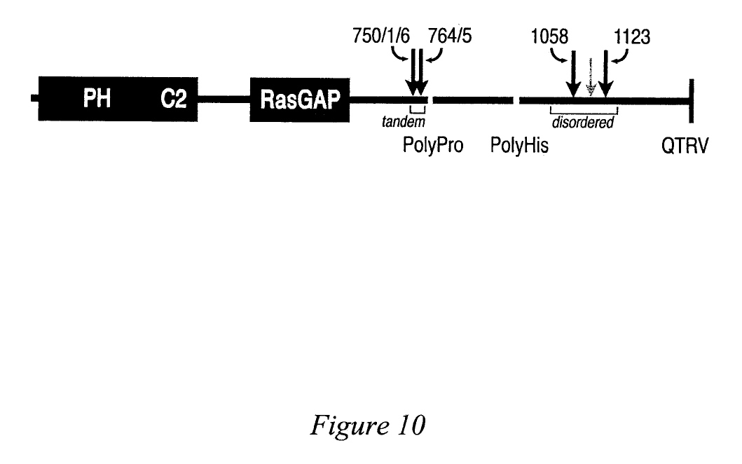

The phosphorylation of synGAP by CaMKII increases its GAP activity by about 70% to about 95%. The increase in activity is observed both when native synGAP present in the PSD fraction is phosphorylated by CaMKII that is endogenous to the PSD fraction, and when recombinant synGAP in Hi-5 cell membranes is phosphorylated by CaMKII purified from rat forebrain. SynGAP is phosphorylated rapidly at several sites in its carboxyl half (FIG. 10), including, serines 751/752 and/or 756, serines 764/765, serine 1058, and serine 1123. FIG. 10 shows a diagram indicating the locations of previously identified domains in the primary sequence of synGAP (Chen et al., 1998; Kim et al., 1998), and the phosphorylation sites described herein. The phosphorylation sites are identified by the large arrows. The location of serines 1093, 1095, and 1099 are indicated by the smaller (curved) arrows. The right bracket indicates the boundaries of the disordered region predicted by the PONDRs software. The left bracket indicates the boundaries of the tandem repeats containing the phosphorylation sites between residues 750 and 765.

As between the two sets of adjacent serines, 751/752/756 and 764/765, it is likely that the adjacent serines may be phosphorylated interchangeably in individual molecules. Experiments with phosphosite-specific antibodies that recognize synGAP when it is phosphorylated at serine 765 or at serine 1123 indicate that phosphorylation of both of these sites is increased in cortical neurons within 15 seconds after activation of NMDA receptors. Whether phosphorylation of these sites in living neurons is catalyzed exclusively by CaMKII remains to be determined. Other sites on synGAP that may also be phosphorylated, but appear from our biochemical results to be less favored, include serines 1093, 1095, and 1099. Mutants in which all of the phosphorylated residues, except serines 751, 752, 756, and 1099, were changed to alanine, singly or in combination were generated. Measurement of GAP activity after phosphorylation of the mutants by CaMKII shows that no single site is responsible for the entire increase in activity. Mutation of serines 764/65 or of serines 1058 and 1123 decreases the level of activation by CaMKII. After mutation of 6 of the identified sites, phosphorylation of CaMKII still increases GAP activity by about 20%. Thus, additional sites, including serines 751, 752, 756, 1099, or other sites later to be discovered, whereby phosphorylation produces a small increase in GAP activity.

Several of the phosphorylation sites that identified herein, including 1058, 1093/95, 1099, and 1123 are located in a region of high disorder spanning residues 1014-1144 (data not shown), as predicted by the software PONDRs [Predictor of Natural Disordered Regions, Pullman, Wash., (Li et al., 2000; Romero et al. 2001; and Li et al. 1999)]. Disordered regions are predicted to be partially or fully unfolded, and, thus, to lack a fixed tertiary structure. PONDRs has been used to detect an association between regions of intrinsic disorder and signaling functions in cell-signaling proteins (Iakoucheva et al., 2002; Dunker et al., 2002). Such regions have also been shown to be involved in DNA recognition, modulation of specificity or affinity of protein binding, and protein regulation (Dyson et al., 2002). The two principal phosphorylation sites, 1058 and 1123, are both immediately preceded by a 13 or 14 amino acid long stretch of polyglycine, broken only by a few serine residues, which is predicted to be maximally flexible. Phosphorylation of sites 1093/95 only in the S1058A and S1123A double mutant were detected. Thus, phosphorylation of these sites may be due to the structural flexibility of this region, permitting phosphorylation of nearby sites in the absence of the favored sites 1058 and 1123.

It was detected that a few weeks after birth, mutant mice in which synGAP has been deleted either from conception, or conditionally, have abnormally high levels of neuronal apoptosis in several brain regions (Knuesel and Kennedy, unpublished data). In addition, preliminary evidence suggests that neurons cultured from mutant mice have alterations in the timing of activation of Ras following activation of NMDA receptors (Manzerra and Kennedy, unpublished data). Knowledge of the phosphorylated sites that produce increased GAP activity of synGAP, and the availability of antibodies that specifically recognize those phosphorylated sites, provides tools to determine the precise roles of synGAP in regulation of synaptic Ras and neuronal apoptosis.

### EXAMPLE 2

### SynGAP Conditional-Knockout Mice Show Increase Cell Death

### Materials and Methods

Generation of Mice with a Floxed Allele of SynGAP

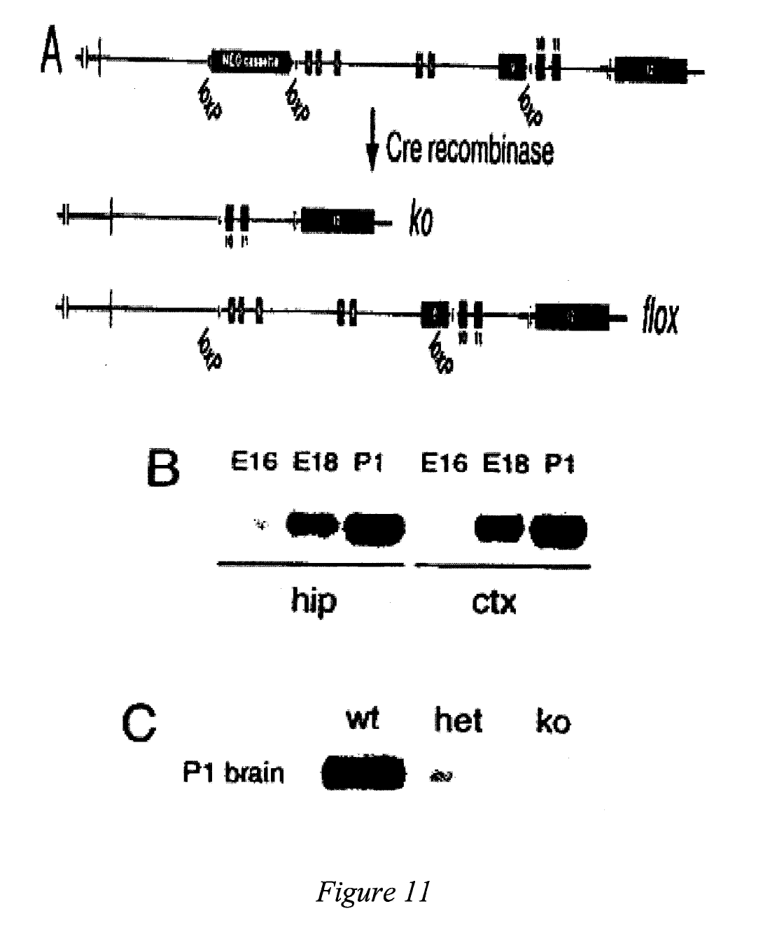

A targeting construct containing exons 4 through 9 and three lox-P sites was constructed and introduced into ES cells as previously described (FIG. 11, Vazquez et al., 2004). The ES cells were transfected with the Cre expressing vector pOG231 (kindly provided by Dr. Henry Lester at Caltech) by electroporation. Twenty eight G418 sensitive clones were identified. Expression of Cre recombinase in recombinant ES cells resulted in either deletion of exons 4-9 of the synGAP gene (ko) or removal of the Neo cassette, leaving two Lox P sites flanking exons 4 through 9 (flox). Two of the clones had complete deletion of sequences between exons 4 and 9 of synGAP (knockout; ko) and the rest had loxP sites flanking the sequences between exons 4 and 9 (floxed; flox). One clone with the flox genotype that had a normal karyotype was used for injection into blastocysts to generate chimeras. Injections and breeding of chimeras were performed by the Transgenic Mouse Core Facility at Caltech. The synGAP flox mutation described here is maintained in a homozygous line and has been back-crossed onto a C57/B6 background. Also, immunoblots showed that all four isoforms of synGAP protein are absent in brain extracts of ko mice and reduced in heterozygote (het) mice at P1 compared to wild-type (wt). Images from sagittal sections taken at P1 showed immunofluorescently labeled synGAP in hippocampal CA1 pyramidal neurons; and punctate staining of synGAP in wt mice was completely absent in ko mice (data not shown). SynGAP ko mice are also smaller in size than wt litter mates on P1 (data not shown). The ko mice are weak, display impaired motor skills and trembling, and generally die by P2. All animal procedures were approved by the California Institute of Technology Animal Care and Use Committee.

Generation of Mice with a Conditional SynGAP-ko Mutation

Excision of the synGAP coding sequence was accomplished by crossing mice bearing the synGAP flox allele with a line of mice expressing a transgene encoding cre recombinase under control of the promoter for the α-subunit of CaMKII (αCaMKII). This promoter drives expression in excitatory forebrain neurons beginning about one week after birth (Schweizer et al., 2003). The most effective reduction of synGAP expression was obtained by creating a line of mice containing the transgene, one flox allele, and one ko allele of synGAP (αCaMKII:cre; synGAPflox/−). To obtain this line, the transgene (αCaMKII:cre) was first bred into a heterozygous synGAP ko background. The resulting αCamKII:cre; synGAP+/− mice were then crossed with the synGAPflox (synGAPflox/flox or synGAPflox/+) line to create conditional synGAP-ko mice (αCamKII:cre; synGAPflox/−), hereafter referred to as cond-ko's. To obtain the initial crosses, the αCaMKII:cre transgene was in a 129vJ background and the synGAP+/− strain had a mixed 129vJ/C57B16 background (the heterozygous ko's breed poorly when fully outcrossed into C57B16.). The synGAPflox gene was outcrossed into a C57B1/6 background.

### Genotyping

Genomic DNA was isolated from mouse tails. The following primers were used to determine the allele of the synGAP gene by PCR: MGIN-11, 5′GAGAGAGATGGAGGGTC ACTTGAG 3′; MGEX9-1, 5′CGGATGCTATGTGCAGTGCTGGA 3′; and Lox-DS; 5′ GAAG AGGAGTTTACGTCCAGCCAAGCT 3′. PCR cycles started with denaturation of DNA at 94° C. for 2 min followed by 35 cycles of the following three conditions: 95° C., 30 sec; 58° C., 30 sec; and 72° C., 2 min; followed by a final extension at 72° C. for 10 min. The PCR products were fractionated on 0.9% agarose gels. A fragment of 1.9 kb indicated the flox allele, 1.8 kb, the wt allele, and 1.7 kb the ko allele. PCR screens for the cre transgene were performed with two primers: Cre-up; 5′CCAGCAACATTTGGGCCAGC 3′ and Cre-low; 5′CGGAAATCCAT CGCTCGACC 3′. PCR cycles started with denaturation of DNA at 95° C. for 2 min followed by 35 cycles of the following three conditions: 95° C., 30 sec; 58° C., 30 sec; and 72° C., 2 min. The presence of the transgene was indicated by a 400 bp PCR product detected on a 2% agarose gel.

Measurement of synGAP by Quantitative Immunoblotting

Brains were collected at embryonic day 16 (E16), E18, P0, P1 (ko line) or at two or eight weeks of age (cond-ko line). Animals at P0 and P1 were anesthetized by hypothermia, and older animals by exposure to an excess of CO₂. Animals were killed by decapitation and the brain rapidly removed. For experiments with cond-ko mice, the right hemisphere was frozen in powdered dry ice and stored at −80° C. until sectioned with a cryostat. Hippocampus and cortex were dissected from the left hemisphere and homogenized at 900 rpm in Teflon-glass homogenizers in RIPA lysis buffer (50 mM Tris, pH 8, 2 mM EDTA, pH 8, 150 mM NaCl, 1% Nonidet P-40, 0.5% Deoxycholate, 0.1% SDS, 0.5 mM DTT) containing a cocktail of protease inhibitors (Complete™, Roche, Nutley, N.J., USA). Protein concentration was determined by the bicinchoninic acid (BCA) method (Pierce, Rockford, Ill., USA) with bovine serum albumin as a standard. Aliquots of protein (10 μg) from cond-ko, mini, and heterozygous control litter mates were loaded in pairs, fractionated by SDS-PAGE, and electrophoretically transferred to PVDF membranes (BioRad Laboratories, Hercules, Calif., USA). Membranes were blocked for 2 h in TBS, 0.1% Tween-20 (TBST), 5% nonfat milk at RT and incubated with rabbit anti-synGAP antibodies (Affinity BioReagents, Golden, Colo., USA, 1:2000) in TBST, 1% NGS ON at 4° C. They were washed three times for 10 min each in TBST, then incubated for 1 h at RT in secondary antibodies (Alexa488, Molecular Probes, Eugene, Oreg., USA, 1:200) diluted in TBST. After three washes in TBST and a rinse with distilled water, membranes were dried and scanned on a STORM™860 scanner (Molecular Dynamics Inc., Sunnyvale, Calif., USA) at 825 volts. The optical density of the protein bands was measured with ImageQuant software (Molecular Dynamics). Two independent sets of gels with duplicate pairs were quantified per sample group.

Immunoblots of brains from synGAP heterozygotes and ko's were performed similarly, except that protein was transferred to nitrocellulose membranes (Schleicher & Schuell, BioScience, Keene, N.H., USA) and bands were visualized by chemiluminescence with SuperSignal® reagents from Pierce.

### Histology and Immunohistochemistry

Embryos at E18, and pups at P0 and P1 (n=2-3 per genotype, from 2-3 different litters) were anesthetized by hypothermia and perfused through the ascending aorta with 4% paraformaldehyde and 15% saturated picric acid in 0.15 M phosphate buffer (pH 7.4). The fixation was preceded by a short rinse with phosphate-buffered saline (PBS; 10 mM NaHPO₄, 120 mM NaCl, pH 7.4). After post fixation for 48 h in the same fixative, brains were transferred to sodium citrate buffer (0.1 M citric acid mixed with 0.2 M Na₂HPO₄ to reach pH 4.5) and incubated over night at room temperature (Fritschy et al., 1998). The tissue was then transferred into 80 ml fresh citrate buffer and irradiated in a microwave oven at medium power for 30 seconds to unmask protein antigens. After cooling down for 15 min, the tissue was rinsed in PBS. Cryoprotection was achieved by immersion in 10% sucrose in PBS for 3 h, followed by 30% sucrose for 24 h. Sagittal sections (50 μm) were cut on a vibratome and stored in antifreeze solution (50 mM phosphate buffer, 15% Glucose, 30% ethylene glycol) at −20° C. until further processing.

Every sixth section (300 μm apart) was Nissl-stained with Cresyl violet to assess histological changes in synGAP ko brains. Changes in protein expression and localization were detected immunohistochemically using rabbit anti-synGAP (Affinity BioReagents, Golden, Colo., USA, 1:500) and rabbit anti-cleaved caspase-3 (Cell Signaling Technology, Beverly, Mass., USA, 1:100) antibodies. To evaluate the specificity of the cleaved caspase-3 antibody reactivity, control sections were incubated with antibody pre-absorbed with blocking peptide (Cell Signaling Technology). Additional controls included incubation with secondary antibodies alone.

Free-floating sections were washed in Tris-saline buffer, TBS (50 mM Tris, 150 mM NaCl, pH 7.4) and preblocked in TBS containing 0.25% Triton X-100 and 10% normal goat serum (NGS) for 30 min at room temperature. Sections were incubated overnight at 4° C. in primary antibody solution diluted in TBS, 0.25% Triton X-100, 4% NGS. Sections were then washed three times for 10 min each in TBS and incubated for 30 min at room temperature in secondary antibodies (goat anti-rabbit Alexa488 or goat anti-mouse Alexa568 [Molecular Probes, 1:500 each]) diluted into TBS, 4% NGS. Cell nuclei were visualized by incubating the slices in 0.25 mg/ml Hoechst 33342 (Molecular Probes) in TBS for 15 min at room temperature. Sections were washed again three times for 10 min in PBS, mounted on poly-L-lysine coated slides (Electron Microscopy Science, Hatfield, Pa., USA) air-dried, and cover-slipped with a drop of ProLong® antifade reagent (Molecular Probes).

Brain sections of the right hemisphere of synGAP cond-ko, mini, and control mice were cut at 15 μm with a cryostat at −20° C., mounted onto poly-L-lysine coated slides (Electron Microscopy Science), and air-dried at RT for 30 seconds. One series of sections per brain was Nissl-stained with Cresyl violet to assess histological changes in mutant brains. Sections used for immunohistochemistry were fixed in methanol at −20° C. for 10 min, transferred into PBS buffer, and processed as described for free-floating sections.

### Image Acquisition and Quantification

A Zeiss Axiovert 200 microscope was used for epifluorescence and light microscopy. Images were acquired with a Plan-NeoFluar 5×/0.15 or Plan-Apochromat 63×/11.4 oil objective and a high-resolution CCD camera (Axiocam MRm, Zeiss, Jena, Germany) under the control of a computer equipped with AxioVision 3.1 (Zeiss). Exposure times were set so that pixel brightness was never saturated, and were held constant during acquisition of all images (1300×1030 pixels) for each experiment. For high-magnification images of fluorescently labeled brain slices, 16 optical sections (“z-sections”; 300 nm intervals) were acquired and deconvolved with the ‘Regularized Inverse Filter’ method (AxioVision software). For visual display and figures, sections were summed and projected in the z-dimension (ImageJ software, NIH, Bethesda, Md., USA).

Quantitative analysis of sections stained for synGAP and activated Caspase-3 were performed on low magnification images of the hippocampus, neocortex, and cerebellum. Brightness of fluorescence was measured in 5 fields (100×100 pixels each) per section in CA1 stratum radiatum or cortex layers I-V and averaged from 6 brain slices per animal (n=4 per genotype) using KS300 software (Zeiss). Data were statistically compared using ANOVA and Tukey Kramer Multiple Comparison test and significance was accepted as p<0.05.

The brightness of the fluorescent staining of activated caspase-3 allowed us to count the labeled cells at low magnification in images (1300×1030 pixels) acquired with a 5× Plan-NeoFluar objective. For every animal (n=2-3 synGAP ko and wild type littermates; n=2-4 mini's, normal cond-ko's and heterozygous littermate controls) six images from six different brain slices were taken from the dorsal hippocampal formation, primary somatosensory cortex, and cerebellum. The number of activated caspase-3 positive cells counted per section in each brain area was averaged and statistically compared between genotypes using unpaired Student's t-test (synGAP ko and wild type) or ANOVA and Tukey Kramer Multiple Comparison test (synGAP cond-ko and heterozygous controls). Significance was accepted as p<0.05. Both image acquisition and quantification were performed blind to the experimental condition.

### Results

Construction of synGAP Knockout and Conditional Knockout Mice

A synGAP knockout mouse line (Vazquez et al., 2004), and a conditional mutant line with a single targeting construct is described herein. The targeting construct contained three loxP sites (FIG. 11A) enclosing a neomycin selection cassette and genomic DNA including exons 4 through 9 of synGAP. After transfection and selection for homologous recombination, mutant ES cells were transfected again with a vector that transiently expresses Cre-recombinase (see Materials and Methods), and screened for one of two mutations: complete deletion of exons 4 through 9 (ko), and introduction of loxP sites surrounding exons 4 through 9 (flox). One ES cell colony bearing the synGAP flox allele was used to generate a line of synGAP flox mice. To delete synGAP in forebrain principal neurons beginning at approximately one week old, mice bearing the synGAP flox allele were crossed with a line of transgenic mice expressing cre recombinase under control of the αCaMKII promoter (αCaMKII:cre, Schweizer et al., 2003). The expression of Cre in the synGAPflox/flox background never reduced the level of synGAP below that of heterozygous ko's, presumably either because of a low rate of recombination and/or a low rate of turnover of synGAP protein. Therefore, the αCaMKII:cre transgene into the synGAP ko background was introduced to produce αCaMKII:cre; synGAP+/− offspring. These offspring were then crossed with synGAPflox/flox mice to produce progeny approximately one quarter of which had the compound heterozygous genotype, αCaMKII:cre; synGAPflox/−. Mice with this genotype exhibited progressive reduction of synGAP protein below levels present in synGAP heterozygotes beginning ˜1 week after birth. A comparison of synGAP ko mice with cond-ko mice is described herein.

Phenotype of synGAP ko Mice

In wild type brains, synGAP expression increases rapidly in the hippocampus and cortex between embryonic day 16 (E16) and postnatal day 1 (P1; FIG. 11B). The synGAP protein comprises at least four major splice variants, all encoded by the same gene (Chen et al., 1998). Immunostaining reveals that it is located in puncta along neuronal somata and dendrites, most prominently in the CA fields of the hippocampus (FIG. 11C). It is expressed at similar levels in all neocortical layers, and in cerebellar Purkinje cells; and at lower levels in olfactory bulb, thalamus, striatum, and brainstem (data not shown). Deletion of synGAP (ko) eliminated all splice variants and eliminated immunostaining in all brain regions at P1 (FIG. 11C). The ko pups appear normal at birth, but do not grow and by P1 become noticeably smaller than their littermates (data not shown). As previously reported, they die before the end of P2 (Vazquez et al., 2004).

### Increased Neuronal Apoptosis in Brains of SynGAP ko Mice

Nissl stained sections of brains from ko and wt mice at P1 revealed no gross anatomical differences in hippocampus and cortex of wt and ko mice. However, the cerebelli of mutant mice were underdeveloped in all 3 animals examined, for example, the cytoarchitecture of the hippocampus and cortex appears normal in ko mice, suggesting a defect in cell signaling during early cerebellar development. Examination of ko brains at higher power revealed more pyknotic nuclei in the CA areas of hippocampus, cortical layers I-VI, and the cerebellar Purkinje cell layer than were present in these brain areas in wt mice of the same age. For example, the cerebellum is underdeveloped in ko mice with reduced folding of the lobes as compared to wt litter mates. Higher magnification images of Nissl stained sections of hippocampal area CA1 reveal many more pyknotic nuclei in stratum pyramidal and stratum oriens of ko mice than of wt controls. Pyknotic nuclei are found in all neocortical layers of ko mice. Less dense cell layers and pyknotic nuclei are found in the Purkinje cell layer and internal granule cell layer of cerebellar sections.

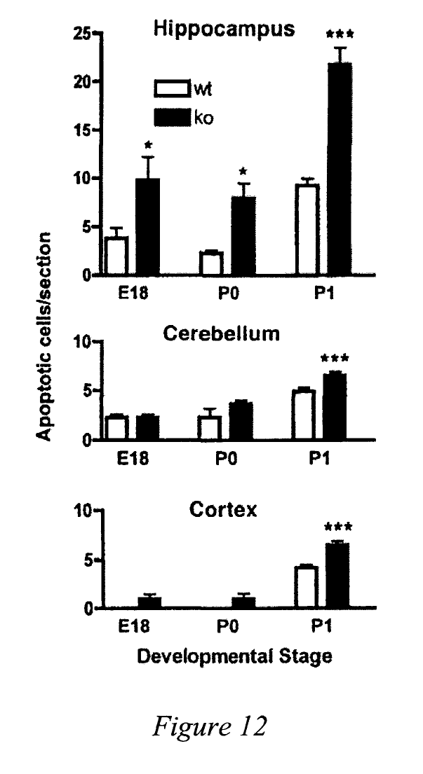

To determine whether the pyknotic nuclei reflect an increase in apoptotic cell death, sections were stained with an antibody against activated caspase-3, a cysteine protease that is a key degradative enzyme in apoptosis (Rami, 2003). The result was that many more neurons stain positively for activated caspase-3 in the brains of synGAP ko mice than in their wild type litter mates. The first significant differences in activated caspase-3 between the genotypes can be seen in the hippocampus at E18 and is also evident at P0 (FIG. 4), a time at which the ko pups are superficially indistinguishable from wt. Immunofluorescent staining with anti-activated Caspase-3 antibodies of sections of the hippocampus showing higher numbers of activated Caspase-3-positive cells per section in ko mice compared to wt littermate controls (low power, images of brain sections at embryonic day 18 (E18), day of birth (P0), and postnatal day 1 (P1) of wt and ko mice; data not shown). Differences are more pronounced on P1 with highest numbers of activated Caspase-3-positive cells in stratum oriens and stratum pyramidale. The data are quantified in FIG. 12. FIG. 12 is a graph showing numbers of apoptotic cells/section, visualized by staining with anti-activated Caspase 3. Activated caspase-3-positive cells were counted in individual optical fields as described under Materials and Methods, and averaged from 4-6 brain sections per animal. Sections from hippocampus, cortical layers I-VI, and cerebellum were counted from two animals at E18 and P0 and 3 animals at P1 for each genotype. Thus, the increased apoptosis is not simply a result of extreme poor health of the mutants. By P1, a significant difference in the number of activated caspase-3-positive neurons between ko and wt mice is also evident in the cerebellum and cortex (FIG. 4).

Higher power images revealed that the activated caspase-3-positive cells have the morphology of neuronal somas in area CA1 in wt and ko (data not shown). The brain were double-stained for cell nuclei (Hoechst 33342, blue) and activated caspase-3 (green). There was a neuronal appearance of activated caspase-3 positive cells and the presence of condensed nuclei in the ko hippocampus. In the hippocampus, most of these apoptotic neurons are found in stratum oriens and the pyramidal layer, whereas stratum radiatum, dentate gyrus, and hilus are nearly devoid of activated caspase-3-positive cells. Apoptotic neurons were distributed throughout cortical layers I-VI and the Purkinje cell layer of the cerebellum (data not shown).

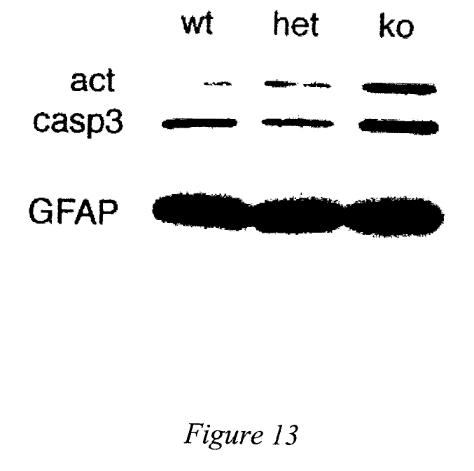

An increase in levels of activated caspase-3 is also visible on immunoblots of homogenates of brains (hippocampal extracts) of homozygous ko's, compared to brains from wt and het mice at P1 (FIG. 13). While levels of activated Caspase-3 are significantly increased in ko mice compared to wt and het mice, GFAP levels remain unchanged in all genotypes. Thus, the presence of abnormally high numbers of neurons undergoing apoptotic death may account, entirely or in part, for the observed weakening and eventual death of the homozygous synGAP ko mutants.

Phenotype of synGAP Cond-ko Mice

Mice heterozygous for synGAP have approximately half as much synGAP in their brains as wt, both at P1 and as adults, but they do not show elevated levels of neuronal apoptosis (data not shown). Whether reduction of synGAP below the level present in heterozygote brains would increase the number of apoptotic neurons in a conditional synGAP ko in which the loss occurs at a stage of development well beyond P1 was explored. To generate such a conditional-ko, a transgene expressing cre-recombinase driven by the αCaMKII promoter was introduced into a synGAPflox/− background, as described above in Materials and Methods. The spatial and temporal pattern of Cre/loxP recombination induced by the αCaMKII:cre transgene line used has been described recently by Schweizer et al. (2003). Cre-induced recombination of an alkaline phosphatase gene in a reporter mouse was first detectable by staining in the hippocampus at postnatal day 17 and then gradually increased to adult levels by P34. Recombination was restricted to principal neurons of the forebrain with highest levels in CA1, dentate gyrus, and cerebral cortex (Schweizer et al., 2003).

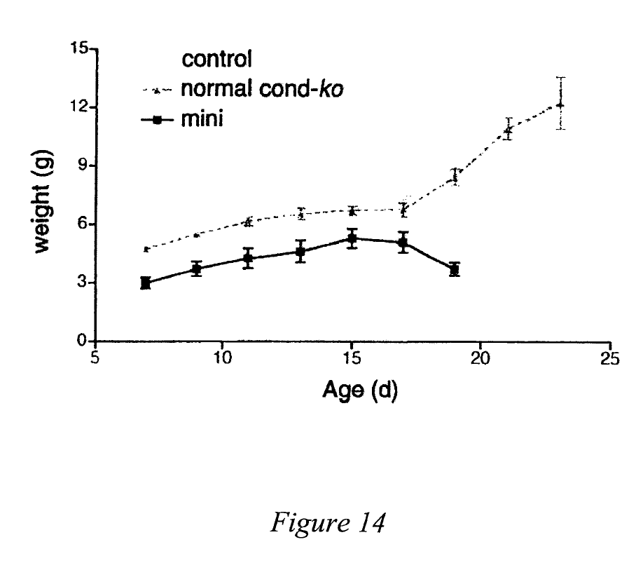

Mice from 7 litters, 3 from crosses of αCaMKII:cre; synGAP+/− with αCaMKII:cre; synGAPflox/+; and 4 from crosses of αCaMKII:cre; synGAP+/− with synGAPflox/+, were individually marked with ink using a micro-tatoo applicator and their weight was recorded every second day from 5 to 28 days after birth. Seventeen (17) pups with the genotype αCaMKII:cre: synGAPflox/− (cond-ko) and 19 littermate controls with genotype synGAPflox/− or αCaMKII:cre: synGAP+/− were followed. Thirteen of the cond-ko mice were phenotypically normal to the fourth postnatal week and their weight was statistically indistinguishable from heterozygous littermate controls. However, 4 of the cond-ko's were significantly smaller at P7. By two weeks after birth, their health started to decline. Signs of distress included lack of locomotion and weight loss. This subset of cond-ko mice as “mini's” and the rest as “normal cond-ko's”. All of the mini's died by three weeks after birth (FIG. 14). Although the normal cond-ko mice remained generally healthy, 4 of the 13 had reduced weight gain after 3 weeks, reflected in the large error bars at day 24 in FIG. 14. In addition, many of the normal cond-ko mice became hyperactive (sometimes running in circles from the top to the bottom of their cage) when attempts were made to handle them or their cages.

Levels of synGAP Protein Correlate with Severity of the Cond-ko Phenotype

Of 103 cond-ko's produced in the colony, 13 (12.6%) have had the mini phenotype. To determine whether the two distinct cond-ko phenotypes are caused by differences in the level of conditional deletion of synGAP, semi-quantitative immunoblots to monitor levels of synGAP protein in progeny of various genotypes was used.

A series of control quantitative immunoblots was performed on homogenates of hippocampus and cortex from 8 week old mice to compare levels of expression of synGAP protein among the following six genotypes (n=2 of each); wt [synGAP+/+], wt plus transgene [αCaMKII:cre; synGAP+/+], synGAP heterozygote [synGAP+/−], homozygous floxed synGAP [synGAPflox/flox], heterozygous floxed synGAP [synGAPflox/−], and heterozygous floxed synGAP plus transgene [αCaMKII:cre; synGAPflox/+]. It was shown that neither cre expression alone, nor the presence of two loxP sites in the synGAP gene, effects levels of expression of synGAP protein. It was also shown that no statistical difference in expression of synGAP between synGAP+/− and synGAPflox/− mice (all p>0.05, ANOVA). Based on these findings, mice of genotypes synGAPflox/− or αCamKII:cre; synGAP+/− were used interchangeably as controls in the following experiments.

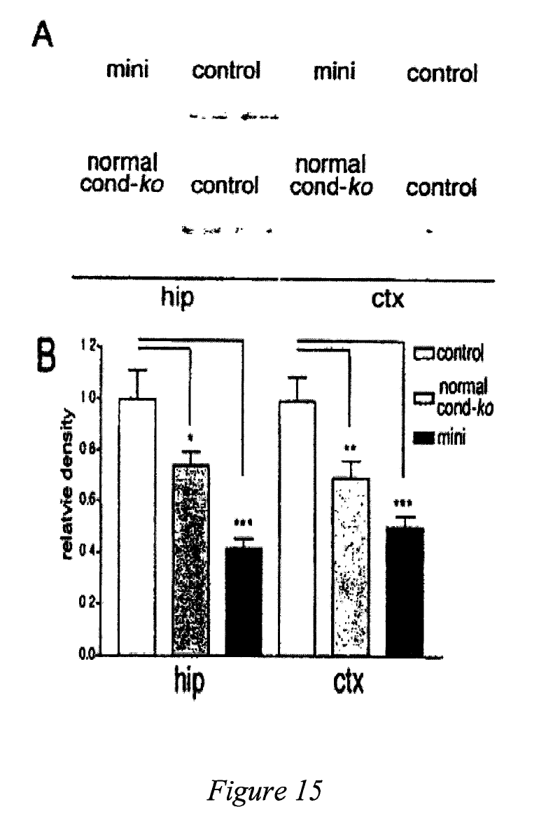

Levels of synGAP at 2 weeks after birth in hippocampus and cortex of 3 mini's, 6 normal cond-ko's, and 12 heterozygous littermate controls were compared. In both brain regions, the level of synGAP in mini's was reduced to 40-50% of heterozygotes, and in normal cond-ko's it was reduced to 75% of heterozygotes (FIG. 15 A, B). In FIG. 15A, representative immunoblots showing various levels of synGAP protein in brains of synGAP cond-ko's and heterozygous controls at two weeks after birth with anti-synGAP antibody of hippocampal and cortical extracts of mini's, normal cond-ko, and control mice. Protein samples were run as duplicates. FIG. 15B is a graph showing that quantitative analysis of immunoblots was carried out as described above in Materials and Methods. Densities of immunoreactive bands from cond-ko's (n=6) and mini's (n=3) were normalized to values for heterozygous controls (n=12; *p<0.05, **p<0.01, ***p<0.001).

These levels correspond to 20-25% of wt in mini's and 37.5% of wt in normal cond-ko's. SynGAP immunoreactivity in fixed brains of normal cond-ko's and mini's was significantly reduced in the dendritic fields compared to heterozygous littermates (FIG. 15C). There were no apparent regional differences in reduction of synGAP between mini's and normal cond-ko's. There was no observable mosaic staining patterns in brains from either genotype. Measurements of the brightness of fluorescent labeling in CA fields and dentate gyrus (data not shown) revealed the same extent of reduction of synGAP levels in each genotype was obtained from analysis of immunoblots. Images of immunofluorescent staining with anti-synGAP antibody of hippocampal slices from 2 week old heterozygous controls and mini's. Levels of synGAP protein in dendritic fields of hippocampi of mini's is greatly reduced compared to heterozygous control litter mates. Thus, it was concluded that 75% loss of synGAP constitutes a threshold below which growth is impaired and the animal eventually dies.

Levels of synGAP Protein Correlate with Severity of Neuronal Apoptosis

To determine whether loss of synGAP can influence neuronal apoptosis when it takes place between 1 and 3 weeks after birth, cells containing activated caspase-3 in brains from synGAP cond-ko mice were counted. Alternating sections from brains of 2 mini's, 2 normal cond-ko's and 2 heterozygote littermate controls fixed at 2 weeks after birth were stained with Nissl stain or immunohistochemically with anti-activated caspase-3 antibodies. The sections were taken from one hemisphere of the same brains that were homogenized for the immunoblot analysis described in FIG. 15 (see also Materials & Methods).

As with ko mice, no gross anatomical differences among the brains of the three groups was found; however, there was significantly more activated caspase-3-positive neurons per brain section in several brain areas of mini's and normal cond-ko's compared to control heterozygous littermates (Table III). Although data are not shown, increased apoptosis in 2 week old synGAP mini's were observed in sections of the dentate gyrus of a 2 week old heterozygous control and a mini double-stained for cell nuclei (Hoechst 33342, blue) and activated caspase-3 (red). There was an increase in activated Caspase-3 cells in the inner third of the granule cell layer in the dentate gyrus of the mini. Also, immunofluorescent staining of 2 week old cortical brain slices from a heterozygous control and a mini were performed. A significant increase in apoptotic neurons is evident in cortical layers II-VI and in the ventricular zone in brains of mini's compared to heterozygous litter mate controls. Quantitative results are shown in Table III below.

Thus, as expected from the expression pattern of cre recombinase conferred by the αCaMKII promoter, more activation of caspase-3 in the cell body layers of the hippocampus (CA stratum pyramidale and DG stratum granular) was found, but not in stratum oriens or stratum radiatum. Although some of the interneurons present in oriens and radiatum express synGAP (Zhang et al., 1999), none of them express CaMKII (Liu & Jones, 1996), and would not be expected to express cre recombinase. This pattern of activated caspase-3 contrasts with the pattern observed in brains from ko animals, which included an increase in active caspase-positive neuronal cell bodies in stratum oriens, as well as in pyramidale (data not shown). In the cerebral cortex, there was significantly more neurons containing activated caspase-3 in layers II-VI and in the ventricular zone in mini's compared to heterozygotes, but not in layer I which lacks positive staining for αCaMKII (Liu & Jones, 1996) (Table III).

TABLE III
Activated Caspase-3 positive cells in sections of brain from heterozygous
controls, normal cond-ko's, and mini's at 2 weeks after birth.

| | | | | Normal | | Mini |
|---|---|---|---|---|---|---|
| | | | | cond-ko | Mini | vs. |
| | | Normal | | vs. | vs. | Normal |
| | Control | cond-ko | Mini | Control | Control | cond-ko |
| CA | | | | | | |
| stratum oriens | 1.0 ± 0.2 | 1.2 ± 0.3 | 0.9 ± 0.3 | n.s. | n.s. | n.s. |
| stratum | 0.4 ± 0.1 | 1.3 ± 0.4 | 1.5 ± 0.3 | P < 0.01 | P < 0.001 | n.s. |
| pyramidale | | | | | | |
| stratum | 2.4 ± 0.4 | 2.3 ± 0.4 | 2.5 ± 0.5 | n.s. | n.s. | n.s. |
| radiatum | | | | | | |
| Dentate Gyrus | | | | | | |
| stratum | 1.4 ± 0.3 | 3.8 ± 0.6 | 12.1 ± 2.0 | P < 0.001 | P < 0.001 | P < 0.001 |
| granulare | | | | | | |
| Cortex | | | | | | |
| layer I | 0.8 ± 0.2 | 0.6 ± 0.2 | 1.1 ± 0.2 | n.s. | n.s. | n.s. |
| layers II-VI | 2.3 ± 0.3 | 2.8 ± 0.4 | 6.9 ± 1.0 | n.s. | P < 0.001 | P < 0.001 |
| ventricular | 1.5 ± 0.2 | 2.1 ± 0.3 | 2.6 ± 0.5 | n.s. | P < 0.05 | n.s. |
| zone | | | | | | |

Counts of activated caspase-3-positive cells in hippocampus and cortex from 6 sections per animal were averaged (number/section ± SEM; n = 4 for heterozygous controls, n = 3 for normal cond-ko's, n = 2 for mini's). P values were determined with unpaired one-tailed Student's t-test. n.s. = not significant.

In two areas, stratum granulare of the dentate gyrus, and layers II-VI of the cortex, mini's have many more neurons containing activated caspase-3 than do normal cond-ko's. Since the mini's also have significantly greater reduction of synGAP expression than cond-ko's, this finding means that the level of induction of apoptosis correlates with the level of reduction of synGAP.

These data show that neuronal apoptosis is increased in conditional synGAP-ko mutants at two weeks after birth, when the level of synGAP protein has begun to fall. The increase in apoptosis occurs only in pyramidal neurons in the forebrain, in which synGAP protein has been reduced due to expression of cre recombinase, and the increase is proportional to the loss of synGAP.

### Increased Apoptosis in 8 Week Old Cond-ko Mice

To determine whether the increased apoptosis in brains of cond-ko mice was restricted to the first few weeks after birth, levels of synGAP and counted neurons containing activated caspase-3 in 4 normal cond-ko and 4 control heterozygote brains from 8 week old mice was measured. Nissl stained sections revealed no histological differences. The reduction of synGAP protein in hippocampi from 8 w old cond-ko's was similar to that from 2 w old cond-ko's, but the reduction in the cortex was not as large (compare FIGS. 15A and B and 16A and B).

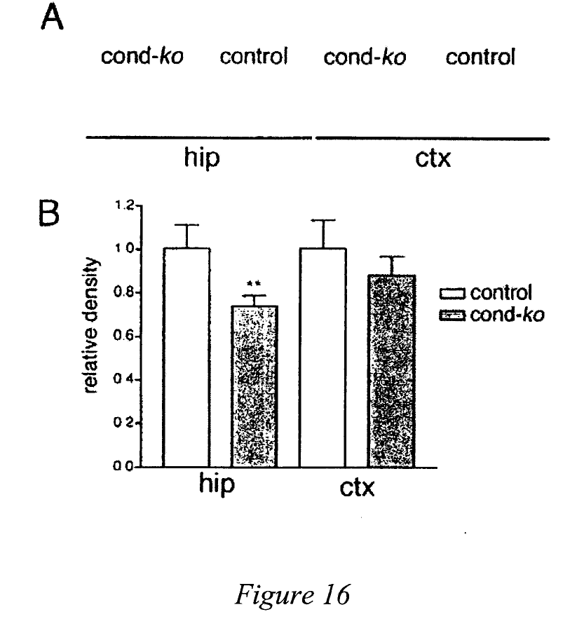

FIG. 16 shows the reduced levels of synGAP protein in 8 week old synGAP cond-ko mice. FIG. 16A shows a representative immunoblot of hippocampal and cortical extracts from heterozygous controls and cond-ko's with anti-synGAP antibody. Protein samples were run as duplicates. FIG. 16B is a graph showing the quantitative analysis of immunoblots reveals a statistically significant difference in levels of synGAP protein in hippocampus but not in cortex of 8 w old normal cond-ko mice compared to heterozygous control litter mates. Densities of bands in normal cond-ko samples (n=4) were normalized to those of heterozygous controls (n=4).

Thus, cre-mediated destruction of the synGAP gene did not increase between 2 and 8 weeks after birth. Caspase-3 activation was significantly increased compared to heterozygous controls in CA stratum pyramidale and DG stratum granulare of the hippocampus; but it was not increased in stratum oriens or radiatum (Table IV).

TABLE IV
Activated Caspase-3 positive cells in sections of brain from heterozygous
controls and normal cond-ko's at 8 weeks after birth.

| | Control | Normal cond-ko |
|---|---|---|
| CA | | |
| stratum oriens | 0.4 ± 0.2 | 0.7 ± 0.2 |
| stratum pyramidale | 0.3 ± 0.1 | 1.3 ± 0.3** |
| stratum radiatum | 1.2 ± 0.2 | 1.4 ± 0.3 |
| Dentate Gyrus | | |
| stratum granulare | 2.3 ± 0.6 | 3.6 ± 0.5* |
| Cortex | | |
| LI | 0.3 ± 0.1 | 0.1 ± 0.1 |
| LII-VI | 1.3 ± 0.3 | 2.4 ± 0.5* |
| ventricular zone | 0.8 ± 0.3 | 0.7 ± 0.3 |

Counts of activated caspase-3-positive cells in hippocampus and cortex from 6 sections per animal were averaged (number/section; mean ± SEM; n = 4 for heterozygous controls and for normal cond-ko's).
*P < 0.05,
**P < 0.01 (unpaired Student's t-test).

In the cortex there was a significant increase in neurons with activated caspase-3 in layers II-VI in 8 w old cond-ko mice compared to controls; but no significant difference in layer I or the ventricular zone (Table IV). The number of layer II-VI neurons containing activated caspase-3 was similar in 2 w and 8 w old cond-ko mice; however, the number of positive neurons in heterozygous controls was significantly lower at 8 weeks. As a result, the difference between cond-ko and control is statistically significant in the 8 w old mice, but not in the 2 w old mice (compare Tables III and IV). The difference between the controls may reflect decreased normal activation of caspase-3 in neurons of adult mice compared to those of 2 w old mice. In summary, levels of synGAP and numbers of neurons with activated caspase-3 were similar in 2 w and 8 w old cond-ko's and showed similar changes in comparison to heterozygous controls (Tables III and IV), consistent with a correlation between the level of synGAP protein and induction of apoptosis.

These data show that the level of neuronal apoptosis, as indicated by activation of caspase-3, is elevated in 8 w old cond-ko mice compared to heterozygote controls. As also observed in 2 week old cond-ko mice, the elevation of activated caspase-3 occurs only in neurons that are expected to express cre recombinase driven by the αCaMKII promoter, and thus to exhibit conditional knockout of the synGAP gene.

### Discussion

The loss of synGAP, a synaptic Ras/Rap GAP protein, causes an abnormally high level of neuronal apoptosis, indicated by the number of neuronal somas containing activated caspase-3. Caspase-3 is an effector caspase that is cleaved and activated by initiator caspases (Budihardjo et al., 1999; Shi, 2002). Its activation commits the cell to apoptosis. The first indication that loss of synGAP increases activation of caspase-3 came from the finding that mouse embryos and newborns with a homozygous knockout of synGAP have increased numbers of neurons in the hippocampus that are stained strongly with antibody against activated caspase-3. It is unlikely that the increased activation of caspase-3 in ko's at this stage is caused by ill health because wt and ko littermates have no obvious differences in size or behavior at birth and both begin to feed normally. By P1, increased activation of caspase-3 becomes visible in neurons in the cerebellum and cortex of ko pups, as well as in the hippocampus. At this time, the ko pups stop growing, begin to weaken, and usually die by the end of the day.

To better establish that deletion of synGAP is a direct cause of the increased activation of caspase-3, conditional synGAP-ko mice that have synGAP at birth, but begin to lose the synGAP gene a few weeks after birth was generated. This cond-ko line carries one copy of the synGAP deletion, one copy of a floxed synGAP gene, and a transgene under control of the αCaMKII promoter which begins to drive expression of cre recombinase at 1 week after birth. When the cond-ko mice were examined at 2 and at 8 weeks old, they had an abnormally large number of neurons staining for activated caspase-3 compared to their wt or heterozygous littermate controls. Furthermore, a greater reduction in synGAP protein correlated with a larger increase in the number of neurons containing activated caspase-3. The level of reduction of synGAP in individual cond-ko's varied considerably. However, mosaicism in the loss of synGAP was not observed in any of the mice. Mice with the most profound loss of synGAP protein (reduced to 20-25% of wild type levels) were reduced in size, died within 2 to 3 weeks after birth, and showed the highest number of neurons with activated caspase-3. These “mini” mice were a minority (12.5%) among the cond-ko's, most of which had a less profound reduction of synGAP protein levels (reduced to ˜40% of wt). Cond-ko's with the less severe reduction of synGAP grew normally for at least 8 weeks; but, nevertheless, had significantly more neurons with activated caspase-3 in their brains than wt.

The location of the neurons containing activated caspase-3 supports the hypothesis that deletion of synGAP acts in a cell autonomous manner. In the ko pups, the highest numbers of activated caspase-3-positive neurons were found in stratum oriens and pyramidale of area CA, the cerebral cortex, and the Purkinje cell layer of the cerebellum. Large numbers of cells with activated caspase-3 appeared very early in stratum oriens and had the morphology of interneurons. GABAergic pioneer cells have been shown to undergo programmed cell death during the first postnatal weeks in wt mice, while others relocate within the hippocampal laminae and differentiate into the interneurons of adulthood (Jiang et al., 2001). SynGAP is expressed in a population of hippocampal interneurons cultured from E18 embryos (Zhang et al., 1999). Thus, loss of synGAP from these neurons may lead to exaggerated programmed death. Apoptotic neurons that appear in stratum pyramidale and in the cerebral cortex have the morphology of excitatory pyramidal neurons. These neurons and Purkinje cells in the cerebellum also express synGAP (data not shown). In the cond-ko, expression of cre recombinase is restricted to excitatory principal neurons in the forebrain by the αCaMKII promoter, leaving expression of synGAP in interneurons intact. Correspondingly, increased activation of caspase-3 was observed only among principal neurons in forebrain regions including stratum pyramidale, cortical layers II-VI, and the dentate gyrus. There were no differences in activated caspase-3 in neurons in CA stratum oriens, radiatum, or cortical layer I, which contain mostly interneurons. Thus, increased numbers of apoptotic neurons are confined to neuronal classes in which the amount of synGAP is reduced.

The effect of loss of synGAP on activation of caspase-3 is unexpected because in adult neurons synGAP is highly concentrated at synaptic sites. However, in developing neurons synGAP is distributed throughout the neuron; and even in adults a small portion appears localized to non-synaptic punctate structures that may be transport vesicles in the soma and dendritic shafts. Thus, synGAP may help to suppress triggering of apoptosis (e.g., actions at synapses, or action of a small portion of synGAP at extra-synaptic sites, and the like). Synaptic synGAP modulating apoptotic pathways is clinically important because Alzheimer's disease, which in its later stages causes severe neuronal death, has early symptoms that can be traced to synaptic dysfunction (Selkoe, 2002).

SynGAP modulates activation of caspases and the onset of neuronal apoptosis by various mechanisms; although the invention is not bound to any one particular theory discussed herein. One mechanism is that synGAP leads to an increase in basal activation of Ras, triggering apoptotic pathways. This general hypothesis is supported by the observations of Henkemeyer et al. (1995) on mice with a targeted deletion of p120-RasGAP, an ubiquitous Ras GTPase-activating protein. The p120-RasGAP ko mice die at embryonic day 10.5 apparently because their vascular systems do not form properly. However, Henkemeyer et al. found extensive apoptotic cell death at E9 and E10 in the branchial arch and several brain regions including the telencephalon. Other tissues appeared relatively free of abnormal cell death. Thus, absence of p120-RasGAP, which is expressed embryonically, causes abnormal neuronal death as early as E9. In contrast to p120 RasGAP, synGAP expression is restricted to the nervous system and begins around E14 to E16 (FIG. 1D), later than that of p120 RasGAP. This may explain why abnormal neuronal apoptosis appears later in synGAP ko's than in the p120 RasGAP ko's.

SynGAP and p120 RasGAP might regulate neuronal apoptotic pathways that are controlled directly by Ras. R-Ras, H-Ras, and oncogenic Ras have been shown to promote apoptosis in cultured cells under particular conditions (Tanaka et al., 1994; Wang et al., 1995; Serrano et al., 1997). Another possibility is that synGAP, like p120 RasGAP, may be a precursor for an anti-apoptotic fragment that is generated by caspase-induced cleavage (Yang & Widmann, 2001; 2002). Yang and Widmann's data show that in situations where caspases are activated at low levels, p120 RasGAP is cleaved into an N- and C-terminal fragment generating a Ras-dependent anti-apoptotic response that prevents the cells from entering apoptosis. Thus, when p120 RasGAP is deleted, developing neurons may progress to full apoptosis more often.

Although the present process has been described with reference to specific details of certain embodiments thereof in the above example, it will be understood that modifications and variations are encompassed within the spirit and scope of the invention. Accordingly, the invention is limited only by the following claims.

### REFERENCES

Annan, R. S., and Carr, S. A. (1996) Anal Chem 68, 3413-3421.
Bayer, K. U., De Koninck, P., Leonard, A. S., Hell, J. W., and Schulman, H. (2001) Nature 411, 801-805.
Bollag, G., and McCormick, F. (1995) Methods Enzymol. 255, 161-170.
Bonni, A., Brunet, A., West, A. E., Datta, S. R., Takasu, M. A. & Greenberg, M. E. (1999). Science, 286, 1358-1362.
Botella, J. A., Kretzschmar, D., Kiermayer, C., Feldmann, P., Hughes, D. A. & Schneuwly, S. (2003) Mol. Biol. Cell, 14, 241-250.
Budihardjo, I., Oliver, H., Lutter, M., Luo, X. & Wang, X. (1999) Annu. Rev. Cell Dev. Biol., 15, 269-290.
Carlin, R. K., Grab, D. J., Cohen, R. S., and Siekevitz, P. (1980) J. Cell Biol. 86, 831-843.
Chen, H.-J., and Kennedy, M. B. (1997) Abstr. Soc. Neurosci. 23, 1466.
Chen, H.-J., Rojas-Soto, M., Oguni, A. & Kennedy, M. B. (1998) Neuron, 20, 895-904.
Cho, K.-O., Hunt, C. A., and Kennedy, M. B. (1992) Neuron 9, 929-942.
Dunker, A. K., Brown, C. J., Lawson, J. D., Iakoucheva, L. M., and Obradovic, Z. (2002) Biochemistry 41, 6573-6582.
Dyson, H. J., and Wright, P. E. (2002) Curr Opin Struct Biol 12, 54-60.
Fritschy, J. M., Weinmann, O., Wenzel, A. & Benke, D. (1998) J. Comp. Neurol., 390, 194-210.
Hellman, U., Wemstedt, C., Gonez, J., and Heldin, C.-H. (1995) Analyt. Biochem. 224, 451-455.
Henkemeyer, M., Rossi, D. J., Holmyard, D. P., Puri, M. C., Mbamalu, G., Harpal, K., Shih, T. S., Jacks, T. & Pawson, T. (1995) Nature, 377, 695-701.
Iakoucheva, L. M., Brown, C. J., Lawson, J. D., Obradovic, Z., and Dunker, A. K. (2002) J Mol Biol 323, 573-584.
Jiang, M., Oliva, A. A. J., Lam, T. & Swann, J. W. (2001) J. Comp. Neurol., 439, 176-192.
Kennedy, M. B. (1997) Trends Neurosci., 20, 264-268.
Kennedy, M. B. (2000) Science, 290, 750-754.
Kim, J. H., Liao, D., Lau, L.-F., and Huganir, R. L. (1998) Neuron 20, 683-691.
Kim, J. H., Lee, H. K., Takarniya, K., and Huganir, R. L. (2003) J Neurosci 23, 1119-1124.
Komiyama, N. H., Watabe, A. M., Carlisle, H. J., Porter, K., Charlesworth, P., Monti, J., Strathdee, D. J., O'Carroll, C. M., Martin, S. J., Morris, R. G., O'Dell, T. J. & Grant, S. G. (2002) J. Neurosci., 22, 9721-9732.
Komiyama, N. H., Watabe, A. M., Carlisle, H. J., Porter, K., Charlesworth, P., Monti, J., Strathdee, D. J., O'Carroll, C. M., Martin, S. J., Morris, R. G., O'Dell, T. J., and Grant, S. G. (2002) J Neurosci 22, 9721-9732.
Kornau, H.-C., Schenker, L. T., Kennedy, M. B., and Seeburg, P. H. (1995) Science 269, 1737-1740.
Kramer, B., Kramer, W., and Fritz, H. J. (1984) Cell 38, 879-887
Krapivinsky, G., Medina, I., Krapivinsky, L., Gapon, S. & Clapham, D. E. (2004) Neuron, 43, 563-574.
Kurada, P. & White, K. (1998) Cell, 95, 319-329.
Lee, A. C., Fenster, B. E., Ito, H., Takeda, K., Bae, N. S., Hirai, T., Yu, Z. X., Ferrans, V. J., Howard, B. H. & Finkel, T. (1999) J. Biol. Chem., 274, 7936-7940.
Leonard, A. S., Lim, I. A., Hemsworth, D. E., Home, M. C., and Hell, J. W. (1999) Proc Natl Acad Sci USA 96, 3239-3244.
Li, X., Romero, P., Rani, M., Dunker, A. K., and Obradovic, Z. (1999) Genome Inform Ser Workshop Genome Inform 10, 30-40.
Li, X., Obradovic, Z., Brown, C. J., Garner, E. C., and Dunker, A. K. (2000) Genome Inform Ser Workshop Genome Inform 11, 172-184.
Lin, A. W., Barradas, M., Stone, J. C., van Aelst, L., Serrano, M. & Lowe, S. W. (1998) Genes Dev., 12, 3008-3019.
Liu, X. B. & Jones, E. G. (1996) Proc. Natl. Acad. Sci. U.S.A., 93, 7332-7336.
Mazzoni, I. E., Said, F. A., Aloyz, R., Miller, F. D. & Kaplan, D. (1999) J. Neurosci., 19, 9716-9727.
McCormick, F., Martin, G. A., Clark, R., Bollag, G., and Polakis, P. (1991) Cold Spring Harb Symp Quant Biol 56, 237-241.
Miller, S. G., and Kennedy, M. B. (1985) J. Biol. Chem. 260, 9039-9046.
Molloy, S. S., and Kennedy, M. B. (1991) Proc. Natl. Acad. Sci. USA. 88, 4756-4760.
Oh, J. S., Chen, H.-J., Rojas-Soto, M., Oguni, A., and Kennedy, M. B. (2002) Neuron 33, 151.
Oh, J. S., Manzerra, P. & Kennedy, M. B. (2004) J. Biol. Chem., 279, 17980-17988.
Peterson, G. L. (1983) Methods in Enzymol. 91, 95-119.
Rami, A. (2003) Neurobiol. Dis., 12, 75-88.
Romero, P., Obradovic, Z., Li, X., Garner, E. C., Brown, C. J., and Dunker, A. K. (2001) Proteins 42, 38-48.
Rose, K., Goldberg, M., and Choi, D. W. (1993) in In Vitro Biological Methods. Methods in Toxicology (Tyson, C., and Frazier, J., eds), pp. 46-60, Academic Press, San Diego
Schweizer, C., Balsiger, S., Bluethmann, H., Mansuy, I. M., Fritschy, J. M., Mohler, H. & Luscher, B. (2003) Mol. Cell. Neurosci., 24, 442-450.
Selkoe, D. J. (2002) Science, 298, 789-791.
Serrano, M., Lin, A. W., McCurrach, M. E., Beach, D. & Lowe, S. W. (1997) Cell, 88, 593-602.
Sheng, M., and Pak, I). T. (2000) Annu Rev Physiol 62, 755-778.
Sheng, M. & Sala, C. (2001) Annu. Rev. Neurosci., 24, 1-29.
Shi, Y. (2002) Mol. Cell, 9, 459-470.
Sjostrom, P. J., and Nelson, S. B. (2002) Curr Opin Neurobiol 12, 305-314.
Smith, D. B., and Corcoran, L. M. (1995) in Curr. Protocols in Molec. Biol. (Ausubel, F. M., Brent, R., Kingston, R. E., Moore, D. D., Seidman, J. G., Smith, J. A., and K., S., eds) Vol. 2, pp. 1671-1677, Wiley Interscience.
Stork, P. J. (2003) Trends Biochem. Sci., 28, 267-275.
Tanaka, N., Ishihara, M., Kitagawa, M., Harada, H., Kimura, T., Matsuyama, T., Lamphier, M. S., Aizawa, S., Mak, T. W. & Taniguchi, T. (1994) Cell, 77, 829-839.
Vazquez, L. E., Chen, H. J., Sokolova, I., Knuesel, I. & Kennedy, M. B. (2004) J. Neurosci., 24, 8796-8805.
Wang, H. G., Millan, J. A., Cox, A. D., Der, C. J., Rapp, U. R., Beck, T., Zha, H. & Reed, J. C. (1995) J. Cell Biol., 129, 1103-1114.
Yang, J. Y. & Widmann, C. (2001) Antiapoptotic signaling generated by caspase-induced cleavage of RasGAP. Mol. Cell. Biol., 16, 5346-5358.
Yang, J. Y. & Widmann, C. (2002) J. Biol. Chem., 277, 14641-14646.
Zhang, W., Vazquez, L., Apperson, M. & Kennedy, M. B. (1999) J. Neurosci., 19, 96-108.
Zhang, W., Apperson, M. L., and Kennedy, M. B. (1997) Abstr. Soc. Neurosci. 23, 1466.

We claim:

1. A method of treating a subject having apoptotic cell death disorder, comprising:
administering to a subject in need thereof, or to cells of a subject, a therapeutically effective amount of an agent that increases expression of Synaptic GTPase-activation protein (synGAP) or increases synGAP activity.

2. The method of claim 1 wherein the cell death occurs in non-dividing cells.

3. The method of claim 1, wherein the disorder is selected from the group consisting of Alzheimer's Disease, Parkinson's Disease, Huntington's Disease and amyotropic lateral sclerosis (ALS).

4. The method of claim 1, wherein administration of the agent increases RasGTPAse activity of synGAP.

5. The method of claim 1, wherein administration of the agent increases phosphorylation of synGAP at serine amino acid positions selected from the group consisting of 750, 751, 764, 765, 1058, 1123 and combinations thereof.

6. The method of claim 1, wherein the subject is mammalian.

7. The method of claim 1, wherein the agent crosses the blood brain barrier.

8. The method of claim 1, wherein the agent is calcium/calmodulin-dependent protein kinase II (CaMKII).

9. The method of claim 1, wherein the agent is a peptide, polypeptide, polynucleotide, or chemical small molecule.

10. A method of ameliorating a pathologic disorder in a subject comprising administering an agent to the subject, wherein the agent increases the expression of synGAP in a cell thereby ameliorating the disorder.

11. The method of claim 10, wherein the degenerative disorder comprises a neurodegenerative disorder.

12. The method of claim 11, wherein the neurodegenerative disorder is selected from the group consisting of Alzheimer's Disease, Parkinson's Disease, Huntington's Disease and amylotropic lateral sclerosis.

13. The method of claim 10, wherein the injury comprises a brain injury or a spinal cord injury.


SEQUENCE LISTING (17 sequences)

SEQ ID NO: 1 - DNA, 24 bp, Artificial sequence
  Primer
gagagagatggagggtcacttgag

SEQ ID NO: 2 - protein, 12 aa, Artificial sequence
  Synthetic construct
MRDLNSSIDLQC

SEQ ID NO: 3 - protein, 12 aa, Artificial sequence
  Synthetic construct
MRDLNSSIDLQC

SEQ ID NO: 4 - protein, 12 aa, Artificial sequence
  Synthetic construct
MRDLNSSIDLQC

SEQ ID NO: 5 - protein, 12 aa, Artificial sequence
  Synthetic construct
SIDLQSFMARGC

SEQ ID NO: 6 - protein, 12 aa, Artificial sequence
  Synthetic construct
SIDLQSFMARGC

SEQ ID NO: 7 - protein, 11 aa, Artificial sequence
  Synthetic construct
RGLNSSMDMAC

SEQ ID NO: 8 - protein, 12 aa, Artificial sequence
  Synthetic construct
RGLNSSMDMARC

SEQ ID NO: 9 - protein, 11 aa, Artificial sequence
  Synthetic construct
RGLNSSMDMAC

SEQ ID NO: 10 - protein, 12 aa, Artificial sequence
  Synthetic construct
LQRGKSQQLTVC

SEQ ID NO: 11 - protein, 11 aa, Artificial sequence
  Synthetic construct
LQRGKQQLTVC

SEQ ID NO: 12 - protein, 12 aa, Artificial sequence
  Synthetic construct
ITKQHSQTPSTC

SEQ ID NO: 13 - protein, 12 aa, Artificial sequence
  Synthetic construct
ITKQHSQTPSTC

SEQ ID NO: 14 - DNA, 23 bp, Artificial sequence
  Primer
cggatgctatgtgcagtgctgga

SEQ ID NO: 15 - DNA, 27 bp, Artificial sequence
  Primer
gaagaggagtttacgtccagccaagct

SEQ ID NO: 16 - DNA, 20 bp, Artificial sequence
  Primer
ccagcaacatttgggccagc

SEQ ID NO: 17 - DNA, 20 bp, Artificial sequence
  Primer
cggaaatccatcgctcgacc